(19) United States
(12) Patent Application Publication
(10) Pub. No.: US 2019/0134188 A1
(43) Pub. Date: May 9, 2019

(54) Recombinant Newcastle Disease Virus Expressing an Immunomodulatory Protein as a Molecular Adiuvant

(71) Applicant: Investigación Aplicada, S.A. De C.V., Puebla (MX)

(72) Inventors: Angel Eduardo Absalón Constantino, Puebla (MX); Diana Verónica Cortés Espinosa, Puebla (MX); Alejandra Galiote Flores, Puebla (MX); Eduardo Lucio Decanini, Puebla (MX); Arnulfo Toscano Contreras, Puebla (MX)

(21) Appl. No.: 16/079,975

(22) PCT Filed: Feb. 24, 2017
(86) PCT No.: PCT/IB2017/051053

§ 371 Date: Aug. 24, 2018

(30) Foreign Application Priority Data
Feb. 24, 2016 (MX) MX/A2016/002399

(51) Int. Cl. A61K 39/215; A61K 39/17; A61P 31/14

(52) U.S. Cl. CPC A61K 39/215; A61K 2039/552; A61P 31/14; A61K 39/17

(57) ABSTRACT

The present invention refers to recombinant Newcastle disease viruses (rNDV) having inserted a transcriptional unit foreign to its genome, which codifies for the synthesis of immunomodulatory proteins. These systems provide excellent protection results and significantly reduce the excreted viral load (post-vaccination and post-challenge) in poultry immunized several weeks after being challenged with a velogenic strain of the Newcastle virus. Additionally, said vaccines allow for the protection of poultry against other pathogenic agents during a long time period as they induce an increase of the immunomodulatory proteins level, which results in an enhancement of the immune response of the host and the development of an efficient humoral and cellular response.

### TECHNICAL FIELD OF THE INVENTION

The present invention relates to the field of veterinary medicine, in particular to the treatment of viral diseases in animals, specifically in the treatment of avian diseases such as Newcastle disease (NDV), among others.

This invention relates to the development of vaccines that enhance the immune response to NDV, more precisely, recombinant Newcastle disease (rNDV) Viruses were generated, bearing an insert of transcriptional unit foreign to its genome, which encodes for the synthesis of Interferon gamma (IFNγ) or Interleukin-6 (IL-6) citokine-type immunomodulatory proteins. These recombinant viruses provide excellent protection results and significantly reduce the excreted viral load in fowls immunized and challenged with the velogenic strain “NDV−P05”. The effect of both recombinant vaccines on the reduction of the viral excretion is attributed to the effect of the cytokines synthesized by the additional genes inserted in the rNDVs genome. This is due to the capacity that both proteins have of targeting and enhancing the immune response of the host, as well as promoting an efficient humoral and cellular response that influences the isotype and the avidity of the antibodies, which finally results in the neutralization of a greater amount of viral particles.

Finally, the recombinant viruses of this application, confer protection against other viral infections additional to the Newcastle disease virus.

### BACKGROUND

Poultry farming is one of the more worldwide developed by man agricultural activities as it develops and fulfills a very important social and economic role. This economic activity has a short exploitation cycle and a good conversion, which allows to provide meat and eggs in a relatively short time period, therefore it plays a strategic role in the feeding of the population, as the poultry products are present in most households because they are nutritive, versatile and maintain relatively low prices (Lopez y Olvera, 2010). The main threat of the poultry industry is the occurrence of diseases caused by bacteria, parasites and viruses, among which there is the Newcastle Disease Virus (NDV) in its virulent form (Aldous and Alexander, 2001; Alexander et al., 2012). The Newcastle disease is caused by a serotype 1-avian Paramixovirus (PMVA-1) that belongs to the Avulavirus genus of the Paramyxoviridae family (King et al., 2012; Mayo, 2002; Murphy et al., 1995). This virus affects more than 250 poultry species, among which there are included domestic poultry such as broiler chickens and laying hens (Alexander et al., 1997; Alexander et al., 2004; Murphy et al., 1999; Rauw et al., 2009). The clinical signs seen in infected poultry range widely from sub-clinical forms, moderate and severe with high mortality depending mainly on the virulence of the circulating strain, the species and the age of the host, the infection with other pathogens, the environmental stress and the immunological condition of the host (Aldous and Alexander, 2001; Alexander et al., 2004; Dortmans, 2011). In every case, the infection with extremely virulent viruses may cause a high and sudden mortality without showing severe clinical signs (Alexander et al., 2004). This disease involves different respiratory, circulatory, gastrointestinal and nervous problems in poultry (Hines and Miller, 2012). These signs basically depends on the age and immune condition of the host and the virulence and tropism of the infecting strain (Alexander et al., 2012; Dortmans, 2011; Dortmans et al., 2011; McFerran and McCracken, 1988). Likewise, the co-infection with other microorganisms and the environmental stress to which the organism is subject are additional factors that aggravate the clinical condition (Aldous and Alexander, 2008; Aldous and Alexander, 2001; Alexander et al., 2004). The following symptoms are commonly observed: Dyspnea, crests and chins cyanosis, muscle spasms, loss of appetite, apathy and indifference, intense thirst, weakness and somnolence (Aldous and Alexander, 2008; Alexander et al., 2004; Dortmans, 2011). At the digestive tract level, one may see an inflammation of the crop, foamy mucus and fibrinous secretion in the pharynx and diarrhea that has a green to yellow shade (Aldous and Alexander, 2008; Carter et al., 2004; Murphy et al., 1999). The nervous signs are expressed by paralysis of wings and legs, torticollis, ataxia or circular movements, clonic spasms and convulsions. In laying poultry there is a drastic reduction of the production of eggs along with depigmentation and loss of the eggshell and a reduction in the quality of albumin (Dortmans, 2011; Dortmans et al., 2011; Murphy et al., 1999). The Newcastle disease virus has an incubation period of 3-6 days depending on multiple factors, including the species of the infected host, the immunity generated towards the NDV and the amount and variety of strains to which poultry is exposed (Bidin and Bidin, 2008; Kapczynski et al., 2013). It is a highly contagious disease with a 100% morbidity in the first 3 or 4 days (Bidin and Bidin, 2008; Kapczynski and King, 2005). The virus is transmitted by utensils, contaminated equipment, personal, other poultries from the farm or wild poultries, and during the acute phase by sprays, food, grains or contaminated water (Alexander et al., 2004; Lopez and Olvera, 2010). The virus may be found in the faeces and is active at low temperatures, however it does not persist with the direct exposure to the Sun. The air serves as the transport for particles bearing the virus, although the spreading in this way is not possible at large distances (Lopez and Olvera, 2010).

In order to keep the disease under control, live vaccines (of lentogenic strains) and inactivated vaccines (Sawant et al., 2011; Seal et al., 2000) are commonly used, which provide good results in the reduction of the clinical condition and in the prevention of mortality (Miller et al., 2007). Nevertheless, it is worth noting that one of the main disadvantages of the prevention of the Newcastle disease, is that the viral excretion (Afonso and Miller, 2013), both of the aetiologic viral agent as well as of the virus used as the vaccine, has not yet been avoided. The viral excretion in poultries of the NDV is given between days 4 and 5 post-infection infection. This excretion leads to a greater likelihood for the virus to circulate among the flock, thus increasing its persistence on the farm. Accordingly, the development of more efficient vaccines that allow for decreasing the spread of the virus reducing the times of viral excretion and improving the immune response to the infection is needed.

The next section will describe the main features of the NDV for the better understating of the invention.

### Classification of the Newcastle Disease Virus

Different genotypes of serotype 1-avian paramyxovirus (APMV-1) circulate in several parts of the world, and although all are recognized as Newcastle Disease Viruses, it is possible to identify a wide genetic diversity among them. Thus, several methods for the classification thereof have been proposed (Absalon et al., 2012; Czegliédi et al., 2006; Miller et al., 2010; Miller et al., 2007). The more recent ones are based on the knowledge and analysis of the full genome of said virus (Absalon et al., 2012), as well as on the analysis with restriction enzymes and the partial or full sequence of the gen codifying the fusion protein (Aldous et al., 2003; Ballagi-Pordany et al., 1996; Diel et al., 2012; Miller et al., 2007). Recently, assessing the genetic diversity of the serotype 1-avian paramyxovirus (APMV-1), Diel et. al. (2012) proposed a nomenclature and a unified classification system based on objective criteria for separating the NDV in different gene groups (Diel et al., 2012).

Newcastle disease virus strains widely vary in terms of the severity of the symptoms that may arise on poultry, and although none of these clinical signs may be considered as pathognomic, certain symptomatologies seem to be associated with some particular kinds of viruses. This has resulted in the grouping of three different pathotypes considering as the baseline those that are predominant in the affected chickens (Alexander et al., 2004; Murphy et al., 1999; Peeters et al., 1999). On this basis, the NDV strains can be:
a) Velogenic: Highly pathogenic form of the disease in which the intestinal hemorrhagic lesions predominate, as well as nerve and respiratory signs. These viruses are characterized by causing high mortality.
b) Mesogenic: virus that cause respiratory and neurological signs, usually cause low mortality.
c) Lentogenic: these type of viruses cause mild respiratory infections.

### Structure and Arrangement of the Viral Genome.

The Newcastle disease virus has the typical structure of a Paramyxovirus, it consists of a lipoprotein coat that surrounds an helical nucleocapsid, it has a single-stranded unsegmented RNA genome and with negative polarity (Römer-Oberdörfer et al., 1999) which may comprise 15,186, 15,192 or 15,198 nucleotides (Dortmans, 2011; Dortmans et al., 2010; Peeters et al., 1999). The genome of this virus (whatever its size is), contains 6 genes: NP-P-M-F-HN-L in the order 3′-5′, which in turn encode for the synthesis of six main structural proteins: The viral nucleoprotein (NP), Phosphoprotein (P), matrix protein (M), fusion protein (F), hemagglutinin-neuraminidase (HN) and the L protein or RNA dependent RNA polymerase (FIG. 1)(Dortmans et al., 2010; Engel-Herbert et al., 2003). Each transcriptional unit has an open reading frame flanked by two non-coding extra-cistronic sequences (UTR's known as “leader” and “trailer” at position 3′ and 5′, respectively. Said regions are followed by conserved transcriptional control sequences known as “gene start” (GS) and “gene end” (GE) which indicate the end (GE) and the beginning (GS) of each open reading frame to the viral polymerase(Kim and Samal, 2010).

These regions are separated by non-coding intergenic regions “Intergenic Sequence” (IS) for which the length thereof has been identified: 1 nucleotide (nt) between NP-P, P-M and M-F; 31 nt between F-HN and of 47 nt between HN-L (Krishnamurthy and Samal, 1998). The modification to this length, increasing it or reducing it, originates dimmed or potentially unworkable viruses (Yan and Samal, 2008). The 6 genes that comprise the NDV genome encode for six main structural proteins, which are schematically represented in FIG. 1 and described below:

### Viral Proteins

### Nucleoprotein (NP).

The nucleoprotein (NP) of the NDV is the largest structural protein since approximately 2,600 copies are estimated for each RNA molecule, to which it binds at all times in such a way that every six nucleotides of the genome interact with a copy of the NP (Egelman et al., 1989). In this way, the NDV genome is never as free as RNA but it is closely associated with the NP protein, which along with the P and L proteins comprise the the ribonucleoprotein complex (Mebatsion et al., 2002; Römer-Oberdörfer et al., 1999). Said complex serves as a mold for the initiation of the transcription and replication of the viral genome (Dortmans et al., 2010), the NP protein being a key factor in the control of these processes, either interacting with the P protein and the L protein or with itself (NP-NP interaction). Furthermore, it has been recognized as a highly immunogenic protein, so it has been used as antigen for diagnosis purposes (Mebatsion et al., 2002).

### Phosphoprotein (P).

Ii is the second more abundant protein of the nucleocapsid with 250-300 copies per virion. It has a molecular weight of 53 000-56 000 Daltons with 1450 nucleotides that encode a 395 amino acids protein (Locke et al., 2000). Its amino terminal end acts as a protective structure that prevents the uncontrolled encapsidation of non-viral RNA by the NP protein (Curran et al., 1995; Mebatsion et al., 2002). As in other paramyxovirus, the edition of the P gene mRNA can be modified during transcription by the addition of Guanine “g” residues in the 484 position, producing alternative ORF's, as a result thereof, at least one additional viral protein called “protein V” (Mebatsion et al., 2003; Mebatsion et al., 2001; Römer-Oberdörfer et al., 1999) is produced, it is speculated about the existence of a second protein called “W” (as in other paramyxoviruses), however its presence has not been confirmed for the NDV (Steward et al., 1993).

The functional versatility of the V protein has been recently investigated and its participation in transcription, synthesis, assembly and viral spread has been shown (Mebatsion et al., 2001; Steward et al., 1993). It has also been confirmed that the V protein is essential for the viral replication and is related with the pathogenicity as it affects the response of interferon and apoptosis in the infected cell (Mebatsion et al., 2003; Mebatsion et al., 2001; Peeters et al., 1999).

### Matrix Protein (M)

The M gene of NDV has a 1241 nt length and encodes for the non glycosylated protein “M” (or matrix) whose molecular weight is 38-40 KD. Said protein forms a peripheral frame or matrix that internally coats the membranous coating, keeping the virion structure when interacting with the cytoplasmic tails of the integral membrane proteins, the lipid bilayer and the nucleocapsid. It plays a key role in the release of the virions of the infected cell since it is considered to be the central organizer of the viral morphogenesis, as its self-association and its affinity to interact with the nucleocapsid is the driving force for the assembly of the viral particle.

### Membrane Glycoproteins

The virus coating consists of a lipoprotein membrane derived from the host cell plasma membrane and modified by the incorporation of the viral transmembrane proteins: Hemagglutinin-neuraminidase (HN) and Fusion Protein (F), both forming lumps on the outer surface of the virus (Huang et al., 2004; Li et al., 2005). F is present on the surface of the virus as an homotrimer, while protein HN exists in its tetrameric form (Lamb et al., 2006; Li et al., 2005; Mirza et al., 1994).

### Fusion Protein (F)

The F protein (or fusion protein) is a type I transmembrane glycoprotein (Lamb et al., 2006) which is initially synthesized as an inactive biologic precursor called F0 consisting of 553 amino acids. In order for it to be biologically functional, F0 shall be divided by host cell proteases on a specific cut site, thus forming two subunits referred to as F1 and F2 (approximate molecular weights of 55 and 12 kDa, respectively), which are kept bond by disulfide bridges (Knipe, 2013; Li et al., 2005). The F protein is found forming homotrimers and is responsible for directing the fusion between the viral membranes and the plasma membranes of the host cell (Li et al., 2005). As a result of this fusion, the nucleocapsid may be released in the cytoplasm, thus starting the replication cycle of the virus. Subsequently, the fusion protein expressed in the plasma membrane may produce the fusion between different adjacent cells forming syncytials or multinucleated giant cells, a cytopathic effect that leads to in vivo tissue necrosis, a possible mechanism for the spread of the virus.

### Hemagglutinin-Neuraminidase (HN).

The HN is a type II transmembrane glycoprotein with its N-terminal end exposed to the cytoplasm, while the C-terminal end is in the lumen of the endomembrane compartment. It is a multifunctional enzyme with hemagglutinating and circumvention activity (Murphy et al., 1999) responsible for the binding of viral particles to the sialic acid receptors of the host cell (Lamb et al., 2006). It further serves as Neuraminidase as it removes such receptors to prevent agglutination (Huang et al., 2004).

The HN sequencing of different isolated NDV's has shown that according depending on the position of the stop codon withing the open reading frame of this gene, three different genotypes of the protein hemagglutinin-neuraminidase may be produced. Accordingly, during the translation 616 proteins of 577 or 571 amino acids may be synthesized. The length is an important factor in the virulence of different strains of NDV (Römer-Oberdörfer et al., 2003). The 616 amino acid proteins are synthesized as an inactive biological precursor called HN0, which needs a proteolytic processing to activate (Garten et al., 1980; Hironori et al., 1987), while those that have 577 or 571 amino acids do not require to be fragmented so as to be functional, being present in most of the NDV strains, including lentogenic, mesogenic and velogenic strains (Römer-Oberdörfer et al., 2003). However, it is important to note that the protein synthesized from HN gene with the shorter ORF (571 amino acids) has only been found in some velogenic strains such as Miyadera and Herts, while the non-virulent strains as Queensland, Ulster and D26 are characterized by particularly producing 616 amino acids proteins (Römer-Oberdörfer et al., 2003).

«L» Protein or RNA dependent RNA polymerase.

Gene L, whose length comprises 6,704 nt, has 6 highly conserved regions considered as essential for the enzymatic activity of the polymerase (Poch et al., 1990), is the most conserved one of the viral genes and the last to be transcribed (Locke et al., 2000; Seal et al., 2000). Its product, the L protein, is a polypeptide whose molecular weight is 242 kDa (Poch et al., 1990). It is the largest of the viral proteins but also the less abundant, and in association with protein P, constitutes the active viral polymerase (Dortmans et al., 2010; Locke et al., 2000; Wise et al., 2004).

### Cell Infection Cycle

The infection cycle begins by the bonding of the virus to the host cell surface. This bonding occurs due to the recognition of specific receptors on the cell surface, which are characterized by exposing sialic acid moieties with which HN protein interacts (Dortmans, 2011; Huang et al., 2004; Lamb et al., 2006). The second step of the infection is mediated by protein F, which is responsible of allowing the penetration of the virus through the induction of the fusion between viral and cell membranes (Li et al., 2005; White et al., 2008). The binding to the HN ligand triggers a conformational change which in turn causes the opening of the F protein and the triggering of a peptide fusion. Thus, F and HN act jointly as a protein assembly to lead the fusion in accurate time and place (White et al., 2008). In the NDV, the binding of HN to the ligand has been proposed as the one responsible for triggering a realignment in its two dimers, wherein a second binding site to sialic acid is exposed, with no sialic acid activity, discovered after the crystallographic analysis of the protein (White et al., 2008). The new formation extends the surface between the dimers, suggesting the release of F of its binding to HN, thus allowing to trigger the fusion. The second HN receptor site maintains the membranes with sufficient proximity as the neuraminidase activity present in the first site does not reside in the second one, which could release the receptor protein and spatially avert one membrane from the other (White et al., 2008).

As a result of this fusion, the nucleocapsid may be released in the cytoplasm and start the virus replication cycle, this mechanism is independent of the pH, although it has been seen that it may become dependent when the fusion process is carried out through the endocytic pathway, as is the case with other paramyxovirus, wherein the pH is critical so as to facilitate the fusion (Cantin et al., 2007; San Roman et al., 1999).

After this fusion process between the two membranes, the nucleocapsid containing the NP encapsulated-RNA genome and associated with the polymerase complex comprised by P and L proteins (Römer-Oberdörfer et al., 1999) enters the cell cytoplasm and begins the transcription to produce the necessary mRNAs for the synthesis of the viral proteins. The binding of the polymerase complex to the nucleocapsid is mediated by protein P, while the catalytic activities are functions of the protein h (Dortmans, 2011).

Throughout this process, the RNA remains bonded to the NP protein and only the mRNAs that encode the proteins of the virus remain free, without binding to the viral proteins. The replication of the viral genome takes place only once the amount of synthesized proteins is enough, then all the components of the virus particle are transported to the plasma membrane where they are assembled under the direction of the M. protein. After the assembly, the viruses are released from the cell through a budding process. The neuraminidase of the HN protein facilitates the detachment of the virus from the cell and eliminates the acid sialic waste in order to avoid the self-aggregation (Dortmans, 2011).

### Vaccination as Control Strategy for the Newcastle Disease (ND).

The vaccination is applied as a preventive measure in many countries of the world and, along to strict biosecurity measures, it has proved to be the most effective method to maintain the ND under control. The principle of vaccination against this disease is well known, in such a way that it seeks to stimulate an immune response that confers protection in the host without this showing the aggressive or lethal consequences of the disease (Lopez and Olvera, 2010). “Hitchner”B1 (HB1) and ‘LaSota’ are the most used vaccine strains around the world, and although more than fifty years have passed since their used started, it is assured that they induce high levels of IgA, IgG, IuM and IgY in the serum of vaccinated poultries, providing protection against mortality and against the more severe clinical symptoms (Borland and Allan, 1980; Russell and Ezeifeka, 1995. Seal et al., 2000). Despite this, the NDV remains a latent risk for populations of broiler chickens and laying hens (Perozo et al., 2006) because despite the intensive vaccination programs, the domestic poultries are still susceptible to suffer severe outbreaks causing a drastic decrease in the production (Absalon et al., 2012; Miller et al., 2007; Rue et al., 2011).

It is widely known that the efficiency of the vaccines used in the current vaccination schemes is limited by the evolutionary dynamics followed by the NDV in various parts of the world. (Diel et al., 2012; Kim et al., 2007; Miller et al., 2010). The difficulty they have to significantly reduce the replication and excretion of the virus, has called the attention for the development of vaccines made with genotypes homologous to the virulent strains circulating in the field. (Hu et al., 2009b; Miller et al., 2013) as on many occasions it has been shown that the heterologous genotype vaccine viruses are less effective in preventing the replication and excretion of the challenge virus (Miller et al., 2009; Miller et al., 2007). For such reason, vaccines homologous to field velogenic genotypes have been developed and are successfully applied in countries that experience significant economic losses because of the NDV (Hu et al., 2009a; IASA-Investigación Aplicada S.A. de C.V.).

### Innate and Specific Immune Response.

The ability of an individual to remain free of infections depends both, on its natural strength or innate immunity as well as on the strength he may develop or acquire during its life (Rojas-Espinosa, 2006). When the organism contacts an external antigen, the immune system immediately responds in an non-specific way. Every element recognized as foreign to the organism causes a migration of phagocytic cells (neutrophils, monocytes, macrophages) which capture the antigen, introduce it into intracellular compartments and destroy it (Rojas-Espinosa, 2006; Wigley and Kaiser, 2003). This mechanism is the responsible of the control of the vast majority of the external agents that the organism faces. A correct operation of these non-specific destruct on mechanisms guarantees, largely, the maintenance of health. Only if this non-specific immunity is not enough to control an infectious process, the organism will seek the activation of the specific defense mechanisms, this is, those that are cell-mediated and which recognize the antigen and specifically respond to it: T and B lymphocytes (Ecco et al., 2011; Kaiser, 2010; Wigley and Kaiser, 2003). The non-specific and specific mechanisms act jointly in space and time communicating between them, both by direct contact between cells as well as by the release of circulating mediators: the cytokines, main mediators and regulators of both types of immune response (Ecco et al., 2011).

### Citoquines

The cytokines are a group with a redundant protein origin and a pleiotropic effect. i.e., they are produced by a variety of lymphoid and non-lymphoid cells and also have several effects on many cells (Kaiser, 2010; Rojas-Espinosa, 2006; Wigley and Kaiser, 2003). The cell sources of cytokines include monocytes/macrophages and dendritic cells, besides T-cells, B-cells, fibroblasts, neutrophils, endothelial cells, primer cells and other. The “cell targets”of various cytokines include, among others, monocytes and macrophages, T cells, B cells, neutrophils, hematopoietic cells of lymphoid and myeloid series, fibroblasts, primed cells and eosinophils (Rojas-Espinosa, 2006). The cytokine effectors include differentiation, proliferation and activation of the “target cells”, the expression of cell receptors for a number of ligands, and the induction and secretion of a variety of other mediators including prostaglandins, cytokines, (growth and differentiation factors, colony formation—stimulating factors), immunoglobulins and others, as well as the release of the same soluble receptors (Rojas-Espinosa, 2006).

### Cytokines as Vaccine Adjuvants.

The cytokines have been classified into a number of groups based on their activity and the cells that are produced by acting accordingly. These groups include interleukins (IL), interferons (IFN), tumor necrosis factors (TNF), transforming growth factors (TGF), inhibitory migration factors, and chemokines (Kaiser, 2010; Wigley and Kaiser, 2003). As a whole, the cytokines are engaged in the induction, expression and modulation of the immune response and in the development and regulation of the inflammatory responses. Their role in mammals is well defined, with a large number of publications which describe the structure of the cytokines and their role in health and disease. While the avian cytokines were poorly defined at the beginning, both in terms of structure and function (Kaiser, 2010). However, in recent years, the advances in the avian immunology and genetics have led to the discovery of a series of cytokines in chicken, turkey and other avian species (Wigley and Kaiser, 2003). This has opened up a wide range of possibilities to determine the levels of cytokines in avian diseases, which allows for a better understanding of the pathogenesis and immunity mechanisms (Ecco et al., 2011), as well as their potential use as vaccine adjuvants or immunomodulators, activating the immune system so that it produces an effective and durable protection (Wigley and Kaiser, 2003).

Several experimental studies have been made using immunomodulators as vaccine adjuvants. A particularly effective method is through recombinant vaccines; wherein the genes encoding the relevant cytokine are inserted into viral expression vectors. Same that, then are used as the vaccine seed (Kaiser, 2010a; Karaca et al., 1998;) (Wigley and Kaiser, 2003). The knowledge and use of avian cytokines as vaccine adjuvants provide a better understanding of the immune response in poultries, which allows to plan more effective control strategies against diseases affecting such organisms (Kaiser., 2010a; Wigley and Kaiser., 2003).

### Recombinant Vaccines

The development of vaccines with better immunogenic and security features is a matter of fundamental importance in the research field of the poultry sector (Alexander et al., 2004; Sawant et al., 2011). In this context, the use of live vaccines using velogenic strains attenuated for its construction/aims to reproduce the infection in a controlled manner and to obtain an immunity similar to that produced by natural infection. However, the confidence for their use is limited by the possibility that exists of producing diseases in immunodeficient poultries or because eventually the attenuated virus may revert to their virulent state (Rojas-Espinosa, 2006).

Despite this it has been shown that the protection is enhanced when it is vaccinated with a strain that is phylogenetically close to the challenge virus al., 2009a; Hu et al., 2011; IASA—Investigación Aplicada S.A. de C.V. et al.), as the humoral and cellular response generated is more intense and long lasting than the one provided by vaccines against inactivated viruses (Absalon et al., 2012; Cornax et al., 2012; Miller et al., 2007; Rue et al., 2011).

Currently, the possibility of enhancing the immunogenicity of vaccines has been possible thanks to the use of the recombinant DNA technology, which allows the isolation of one or more genes that encode for the synthesis of one or more antigenic proteins. The relevant genes are inserted into the genome of the carrier virus through Molecular Biology techniques. Thereby having a recombinant vaccine (Lopez et al., 2003). The inoculation of poultries with these recombinant viruses, leads to the expression or synthesis of the immunizing antigen by the infected cells and immune responses (humoral and cellular) similar to those of the live attenuated virus against the carrier virus and against and the immunizing synthesized antigen (Lopez et al., 2003). In this way, it has been possible to insert genes that express cytokines into the genome of the virus which serves as an expression vector, which play a very important role as modulators of the immune response (Ecco et al., 2011; Kaiser, 2010; Rojas-Espinosa, 2006; Wigley and Kaiser, 2003).

Optimization of the Recombinant Vaccines with Vaccine Adjuvants.

A strategy to improve the immune response stimulated by vaccination is the co-expression (using the vaccine vector) of proteins that play a fundamental role in the processes of induction and activation of the acquired immune response, such as cytokines (Karaca et al., 1998; Lewis et al., 1997). Said proteins accelerate and stimulate the recruitment and activation of accessory cells and the induction of immune response co-stimulators, speeding up its onset and making it more fast, strong and durable (Alvarado et al., 2006.; Manrique, 2005). These proteins serve as powerful immunomodulators acting in different levels due to the need of developing agents that may selectively inhibit or intensify cell populations or subpopulations for the immune response, such as: lymphocytes, macrophages, neutrophils, NK killer and cytotoxic cells (Manrique, 2005). There is an extensive list of cytokines that have been used as vaccine adjuvants in different organisms in order to enhance the immune response, among them we may find: the granulocyte and macrophage stimulating factor (GM-CSF), which helps in recruiting dendritic cells and in increasing the T Cells and B Cells response. Likewise, many Th1 and Th2 type interleukins have been used, such as IL-12, IFN-γ, IL-15 and IL-18, IL-4, IL-10 and IL-13 (Lewis et al., 1997). Particularly, the immunomodulators used in the treatment of avian diseases are listed in Table 1, this Table importantly highlights the use of type I and type II-IFN and of several interleukins in generating vaccines against the Newcastle Disease Virus (Wigley and Kaiser., 2003). The reports suggest their participation in the differentiation and activation of B lymphocytes and T lymphocytes as well as the stimulation of macrophages, so the role they play in the modulation of the immune response before NVD is clear (Ecco et al., 2011).

TABLE 1
Taken and modified from (Kaiser, 2003), it shows the use
that cytokines have as vaccine adjuvant against various avian
diseases; it importantly highlights the use of IFNα and IFNγ as
molecular adjuvants against NDV.
Avian cytokines as vaccine adyuvants before
different pathogens

| | | Etiological | | |
|---|---|---|---|---|
| Cytokine | Species | Agent | Comments | Reference |
| INF-α | Chicken | NDV | The le [symbol] of infection and disease | (Marcus et al., |
| | | | reduce when rINF-a is administered in | 1999) |
| | | | the drinking water. | |
| INF-α | Chicken | NDV | Adjuvant in DNA vaccines using the | (Karaca et al., |
| | | | avian smallpox virus [symbol] expression | 1998). |
| | | | vector. With no clear affect. | |
| INF-α | Turkey | NDV | Adjuvant in DNA vaccines against | (Rautenschlein |
| | | | NDV. Increases the antibodies titers. | et al., 1999a). |
| INF-α | Chicken | TT | The administration of INF-a increases | (Schijns et al., |
| | | IBDV | the titers of tetanus toxoid antibodies | 2000). |
| | | | but not of IBDV | |
| INF-α | Chicken | RSV | Reduction RSV-induced tumors | (Plachy et al., |
| | | | | 1999). |
| INF-γ | Turkey | NDV | With in [symbol] vaccination, the humoral | (Rautenschlein |
| | | | response is faster and provides better | et al., 1990b). |
| | | | protection against the constant | |
| | | | exposition to NDV. | |
| INF-γ | Chicken | Sheep red | Increase of the immune response | (Lowenthal et |
| | | blood cells | | al., 1998). |
| INF-γ | Chicken | NDV | Effecient immune-enhancer used as | (Binjawadagi |
| | | | vaccine adjuvant. | et al., 2009). |
| INF IIpoly | Chicken | MDV | In vitro inhibition of Marek virus | (Heller et al., |
| II | | | replication and deletion of the viral | 1997) |
| | | | proteins encoded in the MDV-infected | |
| | | | cells. | |
| IL-1β | Chicken | TT | Has no effect when administered as | (Schijns et al., |
| | | | adjuvant to TT. | 2000). |
| Linfocinas | Chicken | Enteric | The administration with ILK inhibits the | (Kogul et al., |
| immunes no | | Salmonella | intestinal colonization by Salmonella. | 1997). |
| definidas | | | | |
| (ILK) | | | | |

[symbol]

indicates data missing or illegible when filed

### The Role of IFNγ in the Immune Response Against NDV.

The IFNγ is one of the main soluble protection mediators against viruses since it is released from infected cells almost as soon as the onset, of the infection, i.e., before the antibodies appear. The interferon antiviral action mechanism is not entirely known, but it has been reported that it directly restricts the replication of the viruses at different levels, since penetration, stripping, RNAm synthesis, protein

synthesis and viral particle assembly (Rojas-Espinosa, 2006). “In vitro”e “in vivo”experiments cite it as a powerful macrophages inductor and activator facing the NDV infection, this derives in the regulation of the MHC-II molecules expression which improves the immunity specific against said pathogen (Ecco et al., 2011). When the macrophages are strongly activated, they may damage the normal tissues of the host by releasing the lysosomal enzymes, oxygen and nitric oxide reactive species.

These compounds are effective antimicrobials that lead to the inhibition of the virus replication (reactive peroxynitrite radicals), however, they do not make any distinction between the “Me” and the invading antigens. As a result, if these products enter into the extracellular medium, they are capable of causing tissue damage (Ecco et al., 2011;) Rue et al., 2011).

Also, it has been described that the IFNγ indirectly prevents the viral replication when stimulating the synthesis of the antiviral proteins 2′5′-oligoadenylate synthase and PKR (Double stranded RNA-dependent protein kinase) in the cells that have not yet been infected. The 2′5′-oligoadenylate synthase is an enzyme that synthesizes a 2°15′-oligoadenylate, which is linked to Ribonuclease L (RNase-L) and activates it. The activated RNase-L degrades the viral RNA messengers. On the other hand, the PKR, whose levels are increased by the effect of the viral infection, interfere with the replication of the viral genome with any new infecting virus (Rojas-Espinosa, 2006; Rue et al., 2011).

The role of IL-6 in the immune response against NDV.

Like IFNγ, IL-6 is recognized as one of the main cytokines involved in the regulation of the immune response against NDV (Ecco et al., 2011;) Rue et al., 2011). Originally, IL-6 was identified as a differentiation factor of T lymphocytes and B lymphocytes, among which main functions the stimulation for the production of antibodies being outstanding. Its presence is currently known in local tissue sites, releasing into circulation facing almost all homeostatic disruption situations that typically include endotoxemia, trauma and pathogen-induced intracellular chronic acute infections (Wegenka et al., 1994). It is especially important in the initial phase of the innate immune response, recruiting and activating leukocytes, and setting the stage for she appropriate action in response to the viral infection. It is commonly induced along to other proinflammatory cytokines such as TNF and IL-1 which are necessary for the induction of acute phase reactions in the liver; these include fever, corticosterone release and the hepatic production of protease inhibitor-proteins, which plays a protective role against pathogens (Wegenka et al., 1994). Additionally, it has been described as a potent macrophages, fibroblasts and endothelial cells activator. It improves vascular permeability and stimulates the recruitment of inflammatory cells to the affected site (Ecco et al., 2011;) Wegenka et al., 1994; Wigley and Kaiser., 2003).

In this sense, the administration cytokines as adjuvants during vaccination provides better results than the administration of the vaccine only.

### Objectives of the Invention

Accordingly, an object or the present application is the design and construction of recombinant Newcastle Disease Virus vaccines (rNDVs) having inserted a transcriptional unit foreign to its genome which codifies for the synthesis of the cytokine-type immunomodulatory proteins. (IFNγ or IL-6).

Another object is to show that the rNDVs of the present invention induce the expression of cytokines such as IFNγ or IL-6 in poultries to which the corresponding recombinant viruses were administered.

Another object of the invention is to show that these recombinant viruses increase the protection of poultries.

Another objective of the present invention is to show that the rNDV vaccines significantly reduce the viral load excreted in poultries that were immunized and challenged with the velogenic strain “NDV−P05”. The effect of both recombinant vaccines on the reduction of the viral excretion is attributed to the effect of the cytokines synthesized by the additional genes inserted in the rNDVs genome. This is due to the capacity that both proteins have of targeting and enhancing the immune response of the host, as well as promoting an efficient humoral and cellular response that influences the isotype and the avidity of the antibodies, which finally results in the neutralization of a greater amount of viral particles.

Another objective of this invention is the reduction of the virus excretion after the vaccination/infection process. The ideal is that viruses are not excreted after the death thereof, however, this process occurs during the 6 days following the remission of the disease, to do so, the present invention allows to reduce the excretion of vaccine and pathogen virus, in addition to reducing the exposure time of the animals to the excreted viruses to reduce the infection processes of animals, when having a lower exposure time of the pathogen or vaccine agent, which would solve a health animal problem and would decrease economic costs due to the losses of animals.

Another object of the present invention is that the administration of recombinant Newcastle Disease Virus vaccines to poultries confers protection against other viral infections additional to the Newcastle disease virus for several weeks post-vaccination, these infections may be caused by the Avian Influenza virus, for example. Such protection is set increasing the levels of Interferon and/or Interleukin-6 in poultries several weeks post-vaccination, even in the absence of the recombinant virus.

Another object of the present invention is to increase the levels of cytokines up to 6 weeks post-vaccination, even after the recombinant virus was excreted.

Another object of the present invention is the identification of the recombinant virus with specific oligonucleotides through RT-PCR (Reverse Transcription Polymerase Chain Reaction) to determine if a population of poultries contains the recombinant virus or a native virus and thus diagnose a possible disease or dismiss a false positive.

### SUMMARY OF THE INVENTION

In order to achieve the objects of the present invention, recombinant Newcastle disease viruses (rNDV) were constructed to be used as viral vectors expressing immunomodulator agents (cytokines) from plasma vector which would allow for the insertion of heterologous genes (cytokines) between P and M genes of the Virus, as it has been reported that the heterologous genes introduced between P and M genes have a high level of expression (Nakaya et al., 2001; Zhao et al., 2014). The construction process was carried out based on a transcription vector with low number of copies designated pOLTV5, wherein the full length full length DNAc of both strains (independent constructs) was cloned through the overlapping of sub-genomic moieties previously generated means of the Reverse Transcription Polymerase Chain Reaction (RT-PCR).

The recombinant viruses herein disclosed are originated in the low virulence native strain “LaSota”and the other one in the recombinant strain pRecP05, which was genetically modified to change the aminoacids sequence in the F protein cleavage site, which allows for the virus constructed with pRecP05 to be an attenuated, low-virulence virus).

The construction of the recombinant viruses expressing, separately, the immunomodulator proteins Interferon gamma (INFγ) and Interleukine (IL-6), and the administration thereof to poultries, cause an stimulation of the humoral and cellular immune response due to the production of Interferon gamma or interleukine-6, also enhancing the innate and specific immune response. Such viruses correspond to NVD−LS+IFN and NVD−P05+IFN. Moreover, these recombinant viruses offer 100% protection against mortality facing challenges with field strains.

On the other hand, the inventors show that the vaccination of poultries with the viruses of the present invention has attained to efficiently reduce the amount of particles of the challenge virus excreted to the environment. Comparative studies that quantify the excreted viral load of the challenge virus show that both. “LaSota”as well as the rNDV, are excreted through the sewer up to the 5th d.p.v (post-vaccination day) and through air until the 3rd, however the recombinant strains do this to a lesser extent. The effect on the reduction of the viral excretion is attributed to the IFNγ synthesized by rNDV. This is due to the capacity of IFNγ to target and enhance the immune response of the host, as well as promoting an efficient and sustained humoral and cellular response that finally influences the neutralization of a greater amount of viral particles.

Furthermore, the inventors show that poultries vaccinated with the viruses of the present invention manage to strengthen the immune system of poultries increasing the immune response of poultries against other pathogens such as avian influenza viruses, as a result of the increasing levels of interferon gamma or interleukin-6 during a long post-vaccination period even in the absence of these recombinant viruses with increased levels of IFNγ several weeks post-vaccination.

In addition, the inventors have designed and developed specific oligonucleotides to identify in vitro or ex vivo the heterologous recombinant viruses of the present invention since they selectively amplify the flanking sequence that contains the sites of insertion of the heterologous gene using RT-PCR to identify if a poultries population contains the recombinant virus of the present invention or if it contains some native virus in order to determine if said poultries population has been already immunized.

### BRIEF DESCRIPTION OF THE FIGURES

FIG. 1. Newcastle Disease Virus scheme representing the proteins that are part of the structure thereof.

FIG. 2. Recombinant plasmid map containing the DNAc of the full genome of the “LaSota”strain (pNDVLS).

FIG. 3 pbNANDVII plasmid map wherein the new site recognized by the restriction enzyme NruI may be seen, which was generated by PCR targeted mutagenesis.

FIG. 4. pAENDV2ChIFNγ plasmid map from which it is started to perform the expermental work, this includes sequences with regions Gene end, Intergenic sequence and Gene start (GE, IGS, GS respectively) which flank the genes synthesizing the immunomodulator protein IFNγ (interferon gamma). The full relevant region is strategically delimited by SwaI restriction sites which allowed its extraction to finally clone it in an expression vector.

FIG. 5. Recombinant plasmid map containing the DNAc of the full genome of the “NDV−P05” strain (pRecP05).

FIG. 6. IFN-γ expression in DF-1 cells. Western blot of the supernatant of DF-1 cells wherein the expression of IFN-γ is detected. Lane 1, molecular weight marker. Lane 2, uninfected DF-1 cells. Lane 3, DF-1 cells infected with NDV−P05+IFN virus. The IFN-γ protein has a molecular weight of approximately 17 KDa.

FIG. 7. IFN-γ expression in Vero cells. Western blot of the supernatant of Vero 1 cells wherein the expression of IFN-γ is detected. Lane 1, uninfected cells (control). Lane 2, Vero cells infected with LaSota virus (MOI 0.1). Lane 3, Vero cells infected with NDV−LS+IFN-γ (MOI 0.1). Lane 4, Vero cells infected with NDV−LS+IFN-γ (MOI 1.0). The 17-KDa protein is framed in a rectangle.

FIG. 8. IFNγ auantification by competitive enzymatic immunoassay (ELISA) in vaccinated poultries serum (groups A and B) and not vaccinated (group C). The graph shows a progressive IFNγ increase in poultries immunized with recombinant NDV−LS+IFN vaccine, while poultries immunized with LaSota virus and those non immunized show minimum IFNγ levels over 6 weeks of monitoring.

FIG. 9. Comparative analysis of the macroscopic lesions in internal organs 3 days post-vaccination. A: Poultries immunized with recombinant NDV−LS+IFN vaccine. B: Poultries immunized with LaSota vaccine. C: Control group, non immunized poultries.

FIG. 10. Comparative analysis of the macroscopic lesions in internal organs 5 days post-vaccination. A: Poultries immunized with recombinant NDV−LS+IFNγ vaccine. B: Poultries immunized with LaSota vaccine. C: Control group, non immunized poultries.

FIG. 11. Comparative analysis of the microscopic lesions in internal organs 7 days post-vaccination. A: Poultries immunized with recombinant NDV−LS+IFNγ vaccine. B: Poultries immunized with LaSota vaccine. C: Control group, non immunized poultries.

FIGS. 12A and 12B: Assessment of survival facing challenge with Newcastle disease virus genotype V. The poultries were vaccinated with two different virus, a NDV−LS+IFNγ virus and a LaSota virus (FIG. 12A) and NDV−P05−IFNγ and a LaSota virus (FIG. 12B), in both figures the poultries were challenged with a heterologous genotype V virus. The poultries were challenged three weeks post-vaccination with a lethal Newcastle virus and the survival was monitored during 14 days.

FIGS. 13A and 13B. Excretion of challenge virus in vaccinated poultries. The poultries were vaccinated with NDV−LS+IFN virus or a LaSota virus and a unvaccinated control group. Three weeks after immunization, they were challenged with an heterologous Newcastle virus, genotype V. Samples were taken with tracheal (FIG. 13A) and cloacal swabs (FIG. 13B) at days 3, 5 and 7 after the challenge. The titer of the challenge virus was performed by real time PCR.

FIG. 14. As of the survival facing challenge with Newcastle Disease Virus genotype V. The poultries were vaccinated with two different viruses, a NDV−P05+IFN virus and a LaSota virus and challenged with a Genotype V virus. The poultries were challenged three weeks after vaccination and the survival was monitored during 14 days.

FIGS. 15A and 15B. Excretion of challenge virus in vaccinated poultries. The poultries were vaccinated with a NDV−LS+IFN virus or a LaSota virus and a unvaccinated control group. Three weeks after immunization, they were challenged with an heterologous Newcastle virus, genotype V. Samples were taken with tracheal (FIG. 15A) and cloacal swabs (FIG. 15B) at days 3, 5 and 7 after the challenge. The titer of the challenge virus was performed by real time PCR.

FIG. 16. Specific antibody titer against the avian influenza virus H7N3 before the vaccination (pre-vaccination) and before the challenge (pre-challenge) in groups: Group 1 (NDV−LS+IFN); Group 2 (Emulsión H7N3); Group 3 (NDV−LS+IFN+H7N3 Emulsion) and Group 4 (Control).

FIG. 17. Assessment of the poultries survival facing challenge with high virulence influenza A virus. Poultries were vaccinated with different vaccines: NDV−LS+IFN, a dual vaccunation with NDV−LS+IFN+H7 Emulsion, H7 Emulsion and a unvaccinated control group. Poultries were challenged with an influenza virus three weeks post-vaccination and the survival was monitored for 14 days.

FIGS. 18A and 18B. Excretion of challenge virus in vaccinated Poultries. The poultries were vaccinated with a. NDV−LS+IFN virus +H7N3 Emulsion, H7N3 Emulsion, NDV−LS+IFN and a unvaccinated control group. Three weeks after immunization, they were challenged with an heterologous Newcastle virus, genotype V. Samples were taken with tracheal (FIG. 18A) and cloacal swabs (FIG. 18B) at days 3, 5 and 7 after the challenge. The titer of the challenge virus was performed by real time PCR.

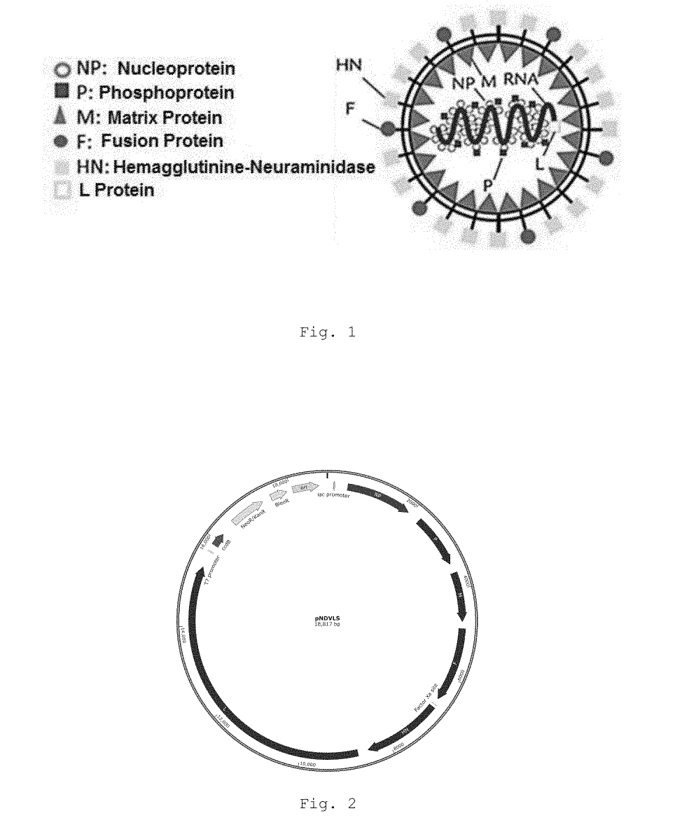

### DETAILED DESCRIPTION OF THE INVENTION

The present invention consists of various Newcastle disease virus constructed through reverse genetic and molecular biology techniques, which have the capacity of expressing the Interferon gamma gene (IFN-γ) and the Interleukin-6 gene of the species Gallus gallus. The recombinant virus herein described contain molecular signals that allow the transcription of the Messenger RNA of the IFN-γ gene by the RNA dependent RNA polymerase of the Newcastle disease virus. In addition, it contains a molecular signal that allows it to be translated into the full and active IFN-γ protein in eukaryote cells.

The recombinant viruses contain the sequences that encode for one or more of the NP, P, F, HN and L proteins of the Newcastle disease virus which are useful for the development of live vaccines and can additionally be used as vectors for the incorporation of proteins of other types of heterologous viruses (WO99/66045).

### EXAMPLE 1

### Construction of Recombinant Virus Expressing IFN-γ based on LaSota Strain (NDV−LS−IFN).

The construction of the recombinant virus based on the strain LaSota was performed by reverse genetic methods (Peeters et al., 1999). To this end, 8 segments were amplified by means of RT-PCR using overlapped oligonucleotides of one clone of the LaSota strain. The 8 segments were cloned in autonomous replication plasmids and later bonded using restriction and molecular cloning enzymes until having the full genome of the virus (FIG. 2).

At the same time, a plasmid containing the segment ApaI-NotI of the NDV genome LaSota strain called pNDVApa-Not was constructed. Using this plasmid as a mold, targeted mutagenesis was performed using the Phusion Site-Directed Mutagenesis kit system (Thermo Scientific) to create a cleavage restriction site for the NruI enzyme in the non-coding region 5′ (NCR) of the P gene of the NDV encoded in the negative chain of the virus genome. The new plasmid obtained with the NruI restriction site was called pNDVANII (FIG. 3).

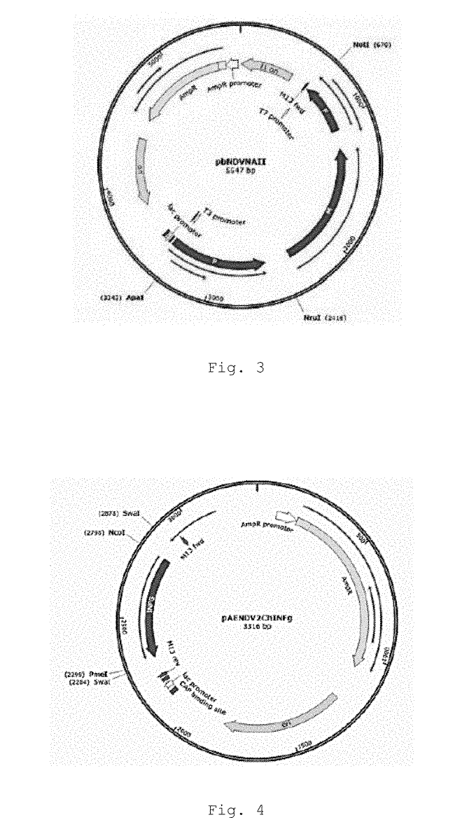

On the other hand, a plasmid containing the signals for the RNA polymerasa of the NDV of the start (GS) and the end of the transcription was constructed, separated by a nucleotide serving as intergenic sequence (IG). Furthermore, this plasmid included recognition sites for restriction enzymes that allow to clone a gene in the region 5′ NCR in the negative chain of the virus genome. All these synthetic sequences are bounded by two restriction sites for the NruI enzyme. The plasmid containing the synthetic sequence GE-IG-GS was calling pAENDVII. Using the plasmid pAENDVII, the cloning of the gene encoding for chicken interferon gamma (IFN-γ) (Gallus gallus) was performed, obtaining pAENDVIFN (FIG. 4).

Subsequently, the NruI segment of the pAENDVIFN plasmid was cloned in the restriction site NruI of the plasmid pNDVANII, to form pNDVANIFN. Finally, the plasmid pNDVANIFN was digested in double digestion with ApaI-NotI and cloned into the plasmid pNDVLS.

The nucleotide sequence of the obtained recombinant virus corresponds to SEQ ID NO. 2, the length of said virus is of 15780 bases and contains the heterologous gene that encodes for the IFNγ of the species Gallus gallus (chiIFN-γ).

For the rescue of the virus, the transfection of the plasmids pNDVIFN γ, pNP, pP and pL was made within the cell line Hep2 previously infected with the virus Vaccinia MVA-T7 Ankara.

After transfection, the cells were maintained for 72 hours in MEM medium added with 5% fetal bovine serum and incubated at 37° C./5% CO₂ until their analysis.

After 72 hours, the supernatant of the transfected cells was used to inoculate 9 days old Specific Pagthogens Free (SPF) Chicken embryos. Four days later the allantoic fluid was collected and analyzed with an hemagglutination assay (HA) using 5% chicken erythrocytes to corroborate the presence of virus.

### EXAMPLE 2

### Construction of Recombinant Virus Expressing IFN-γ based on Genotype V NDV Virus (NDV−P05−IFN)

The second recombinant virus expressing the gene that encodes for IFN-γ protein was design based on an NDV skeleton belonging to class II and particularly the genotype V and sub-genotype Vb. This recombinant virus was 100% constructed using chemical synthesis using the sequence of the virus APMV1/chicken/Mexico/P05/2005 as the mold.

The skeleton of the recombinant virus called A/Synthetic/RecP05−IFN/2013 (short name P05−IFN) was obtained from 3 segments of double-stranded DNA chemically synthesized from approximately 5-6 kb which were cloned in plasmids which gave rise to pRecP05-H1 pRecP05-H2 and P05-H3. The skeleton of the synthetic virus in the form of complementary DNA can be used as a vector for the insertion of homologous heterologous genes to the NDV.

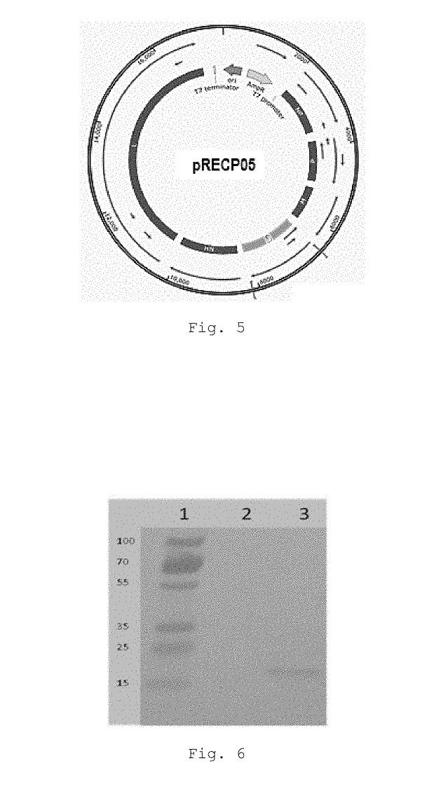

The construction of the synthetic recombinant virus was determined by the assembly of the three 3 synthetic segments using restriction and ligation enzymes as shown in FIG. 5.

The nucleotide sequence of this recombinant virus corresponds to SEQ ID NO. 1, the length of said virus is of 15780 bases and contains the heterologous gene that encodes for the IFNγ of the species Gallus gallus (chiIFN-γ).

The recovery of the virus was carried out according to the protocol described in Example 1.

### EXAMPLE 3

### Assessment of IFN-γ Expression in the Recombinant Viruses NDV−LS−IFN and NDV−P05−IFN.

Once both recombinant virus obtained in chicken embryos, we proceeded to prove that both viruses have the ability to express the interferon-γ (IFN-γ) gene encoded in the viral genome. For this ends, a Western Blot assay was performed using DF-1 and Vero cells cultures. DF-1 cells are chicken embryo fibroblasts not expressing IFN-γ. Vero cells are green monkey kidney epithelial cells not expressing IFN-γ chicken gen. Both cell lines are susceptible of being infected by the Newcastle virus.

DF-1 cells were exposed to two treatments, the first flask was infected with the NDV−P05+IFN virus and another flask was not infected to validate that the DF-1 cells used do not express chIFN-γ. The cells were incubated for 60 hours to allow for the expression of the protein and the supernatant was sampled.

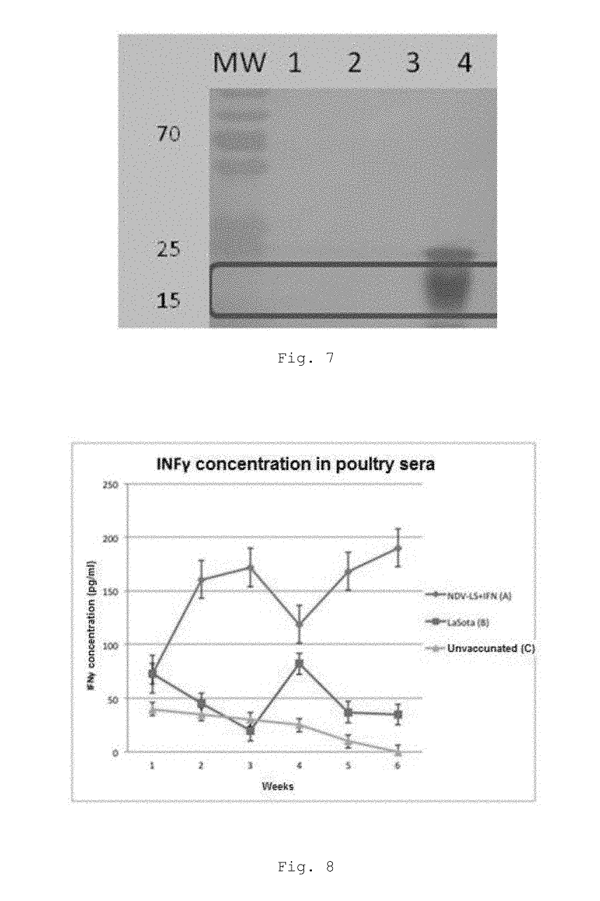

Our results confirm that only the sample of the flask infected with the vaccine virus NDV−P05+IFN expressed the IFN-γ. This was identified as a protein of approximately 17 kDa, as it may be seen in the western blot shown in FIG. 6.

In a second experiment, Vero cells were used. The Vero cells were exposed to three treatments, the first flask was infected with the NDV−P05+IFN virus, the second flask was infected only with a single LaSota virus and another flask was not infected to validate that the VERO cells used do not express IFN. The cells were incubated for 40 hours and the supernatant s sampled.

The samples of the Vero cells supernatant were subjected to western blot assay to identify the chIFN-γ contained in the supernatant, as shown in FIG. 7. The results obtained confirm that the sample of the flask infected with vaccine virus NDV−LS+IFN (MOI 1.0) expressed chIFN-γ, as it may be seen in lane 4 of FIG. 7. This protein was identified as a protein approximately 17 kDa.

In addition, each of the treatments were sampled to evaluate, by real time RT-PCR, the amount of the chIFN-γ messenger RNA using the corresponding oligonucleotides. Our results indicated that the non-infected Vero cells were negative both for Newcastle as well as for chIFN-γ. On the other hand, the flask infected with the LaSota virus was only positive for the Newcastle disease virus (titer of 1.234*10⁹ copies per ml). Finally, the flask infected with the NDV−LS+IFN virus was positive both for Newcastle (titer of 1294*10⁹ copies per ml) and for chIFN-γ (titer of 1.754*10⁸ copies by ml). These results are shown in Table 2. These experiments confirm that the gene exists, that there is transcription and translation to a 17 kDa protein that is recognized by an anti chIFN-γ antibody.

TABLE 2
Titers of the chIFN-γ messenger RNA measured by real time
RT-PCR in nonVero cells samples non-infected and infected with LaSota
and NDV-LS + IFN virus.

| | Newcastle Titer | chIFN-γ Titer |
|---|---|---|
| Treatment | (copies per ml) | (copies per ml) |
| Uninfected Vero | NEGATIVE | NEGATIVE |
| Cells | | |
| Vero Cells | 1.234 * 10\hat{ }9 | NEGATIVE |
| infected with | | |
| LaSota virus | | |
| Vero Cells | 1.294 * 10\hat{ }9 | 1.754 * 10\hat{ }8 |
| infected with | | |
| NDV-LS + IFN | | |

These results as a hole indicate that the heterologous gene encoding for chIFN-γ introduced in the NDV LaSota strain and P05 strain encodes a protein of approximately 17 kDa corresponding to chicken interferon gamma, according to the results shown in the immunoblots of FIGS. 6 and 7.

On the other hand, these results show that cells that are transfected with the described recombinant viruses secrete considerable amounts of chicken interferon gamma into the extracelular media, this is shown by the results obtained from the supernatant of Vero cells in FIG. 7.

In addition, this sample demonstrates “in vitro” the chIFN-γ expression as a result of the infection of NDV−LS−IFN and NDV−P05−IFN recombinant heterologous viruses in a cell model that does constitutively express the chicken interferon gamma.

### EXAMPLE 4

### Assessment of the Presence of IFNγ in Vaccinated Poultries

In this example we proceeded to determine the “in vivo” expression of IFN-γ in Gallus gallus. For this end we used 40-3 weeks old specific pathogen free (SPF) light poultry. 3 groups were generated. Group 1 was not vaccinated (negative control),

Group 2 was vaccinated with a LaSota strain virus to determine the induction of the IFN-γ due to this vaccine strain and Group 3 was vaccinated with the recombinant virus NDV−LS+IFN.

Monitoring was made for 6 weeks after vaccination and the

ELISA test was used to quantify the amount of IFN in sera from vaccinated poultries.

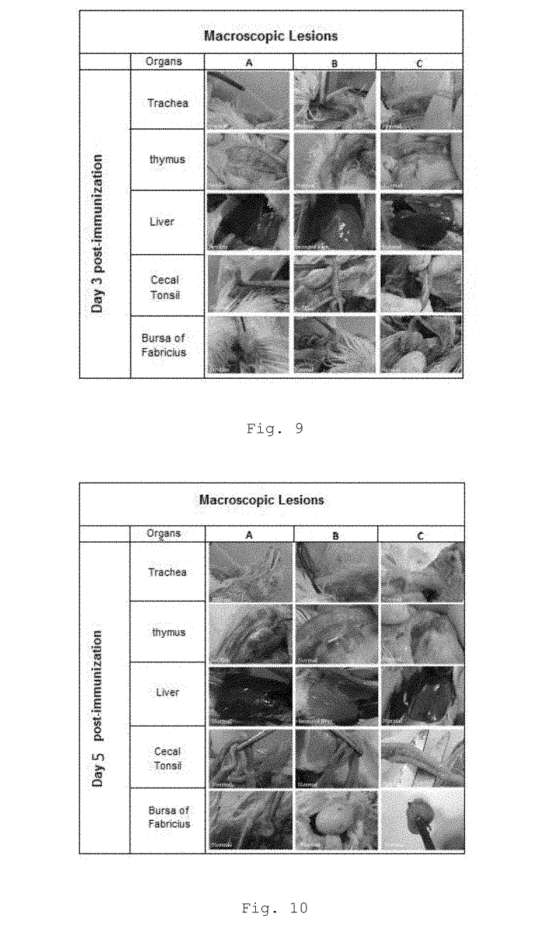

The results of FIG. 8 indicate that the vaccine containing the recombinant virus NDV−LS+IFN induces an increase in IFN-γ in the host, increase that is relevant from the second week post-vaccination.

The poultries vaccinated with NDV−LS+IFN show an increase in the IFN-γ concentration during the 6 weeks of the experiment. In week 2, there is recorded a drastic increase in the IFN-γ concentration in poultries vaccinated with NDV−LS+IFN. This increase remains high during the 6 weeks of monitoring, although there is a decline registered in week 4. However, it is clear that from week 2, the poultries vaccinated with the NDV−LS+IFN virus have higher IFN-γ concentration than the poultries vaccinated with LaSota virus and the unvaccinated poultries. In the case of poultries vaccinated with LaSota viruses, there is an irregular behavior in the IFN-γ expression having a peak after 4 weeks, which is still below the IFN-γ value of poultries vaccinated with the NDV−LS−IFN virus. In the case of unvaccinated poultries, the decrease in the production of IFSγ is gradual until it reaches close to zero values after 6 weeks post-vaccination. This is clearly shown in FIG. 8.

From these results, it is inferred that the vaccination of poultries with the recombinant virus NDV−LS−IFN permanently raises the interferon gamma levels at least during 6 weeks, and that is not the case for poultries vaccinated with LaSota virus or in the unvaccinated poultries.

It is important to remember that the excretion of the virus, whether recombinant or pathogen, appears until maximum the seventh day post-vaccination or post-infection, therefore one should expect that in the case of the recombinant viruses of the present invention, the IFN-γ expression should be reduced after the sixth day post-infection.

However, from the experiment shown in FIG. 8 deduced that the presence of IFN-γ in the sera samples of the poultries in the late weeks of this experiment (4-6 weeks post-vaccination), is not due to the presence of the virus as the excretion of the challenge virus is made after 3 to 7 days of the infection (as will be seen below in Examples 5, 6 and 7); this result is unexpected and novel as instead of reducing the INF-γ levels due to the reduction of the levels in the recombinant viruses, we found an increase in the constitutive expression of IFN-γ in poultries, which remains constant until the end of this experiment.

This effect is not present in poultries vaccinated with LaSota virus, wherein although it is true that the vaccine virus induces INF-γ expression in poultries, the amount of this cytokine is considerably reduced at the level of the unvaccinated poultries in week 4.

This shows, undoubtedly, that, unexpectedly, the recombinant virus NDV−LS+IFNγ is capable of inducing the constitutive expression of IFN-γ in poultries. This means, in the case of broiler chicken, that the INF-γ expression is kept during the entire life of poultries, which would induce an immune protection during all the time the poultries remain in the farm as the mean life span of the broiler chicken is of 5 to 6 weeks, thus, it may be inferred that the poultries keep a cellular immune response in the form of INF-γ during all their lives in the farm.

### EXAMPLE 5

### Assessment of LaSota recombinant vaccine (NDV−LS+IFN)

In another example of the present invention, there is provided that the NDV−LS−IFN virus, whose construction is described in Example 1, was used as the seed for the development of a “live” lyophilized vaccine and the protection under a challenge of a high virulence strain of the Newcastle disease virus was assessed. The lyophilized vaccine was formulated at a final concentration of 1×10⁶ viral particles/dose.

3 groups of poultries each containing 22 poultries were made for this experiment. Group A was vaccinated with NDV−LS−IFN virus; Group C was vaccinated with LaSota strain and Group D was not vaccinated. From these poultries, 9 poultries from each group were sacrificed in days 3, 5 and 7 of vaccination (3 daily) to assess the damages in lymphoid organs and trachea due to the vaccine virus. In addition, other 3 poultries in each group were sacrificed in the 5th day post-challenge (to assess the damages in internal organs caused by the challenge virus. The remaining 10 poultries were used to measure the survival to the challenge.

Assessment of the post-vaccination effect on poultries.

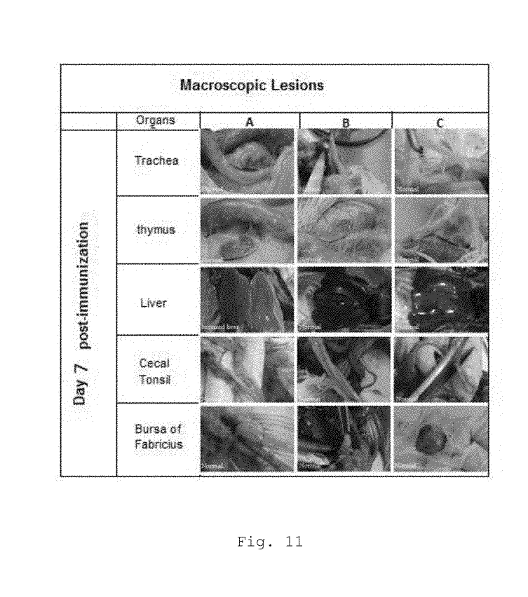

In order to assess the lesions caused by the vaccine virus, the lymphoid organs and trachea were sampled in poultries at days 3, 5 and 7 post-vaccination (FIGS. 9, 10 and 11). 3 groups of poultries each containing 9 poultries were realized for this experiment. Group A was vaccinated with NDV−LS−IFN virus; Group B was vaccinated with LaSota strain and Group D was not vaccinated.

The images in FIG. 9 show that no lesions were detected in vaccinated poultries as compared with control poultries (unvaccinated) and with poultries vaccinated with LaSota virus 3 days post-immunization.

The only noticeable difference is that the poultries vaccinated with the NDV−LS+IFN virus show, in general, an enlargement of the thymus; possibly due to the high interferon-γ concentration that the recombinant virus itself produces.

Table 3 shows the summary of the data of representative FIGS. 9, 10 and 11, wherein the lesions found after applying treatments A, B and C, NDV−LS+IFN-γ vaccine, LaSota virus and non-immunized poultries, respectively.

TABLE 3
Comparative analysis of the macroscopic lesions in internal organs
at days 3, 5 and 7 post-vaccination. A (NDV-LS + IFN-γ Vaccine),
B (LaSota virus) and C (non-immunized poultries). The signs (+)
indicate that there was some damage in the assessed tissue. Summary
of FIGS. 9, 10 and 11.
MACROSCOPIC LESIONS POST-IMMUNIZATION

| | Day 3 | | | Day 5 | | | Day 7 | | |
|---|---|---|---|---|---|---|---|---|---|
| Poultries/Organ | A | B | D | A | B | C | A | B | C |
| Trachea | [symbol] | [symbol] | [symbol] | [symbol] | [symbol] | [symbol] | [symbol] | [symbol] | [symbol] |
| Thymus | [symbol] | [symbol] | [symbol] | [symbol] | [symbol] | [symbol] | [symbol] | [symbol] | [symbol] |
| Liver | [symbol] | [symbol] | [symbol] | [symbol] | [symbol] | [symbol] | [symbol] | [symbol] | [symbol] |
| Lungs | [symbol] | [symbol] | [symbol] | [symbol] | [symbol] | [symbol] | [symbol] | [symbol] | [symbol] |
| Cecal tonsil | [symbol] | [symbol] | [symbol] | [symbol] | [symbol] | [symbol] | [symbol] | [symbol] | [symbol] |
| Burst | [symbol] | [symbol] | [symbol] | [symbol] | [symbol] | [symbol] | [symbol] | [symbol] | [symbol] |
| Spleen | [symbol] | [symbol] | [symbol] | [symbol] | [symbol] | [symbol] | [symbol] | [symbol] | [symbol] |
| Mortality | 0 | | | 0 | | | 0 | | |

[symbol]

indicates data missing or illegible when filed

These results indicate that the recombinant virus IFN−LS−NDV induces no macroscopic lesions post-vaccination, therefore the virus is secure because it does not cause any lesion in the vaccinated poultries.

### EXAMPLE 6

### Assessment of the Protection of the NDV−LS+IFN Vaccine to the Challenge with Velogenic Newcastle Virus

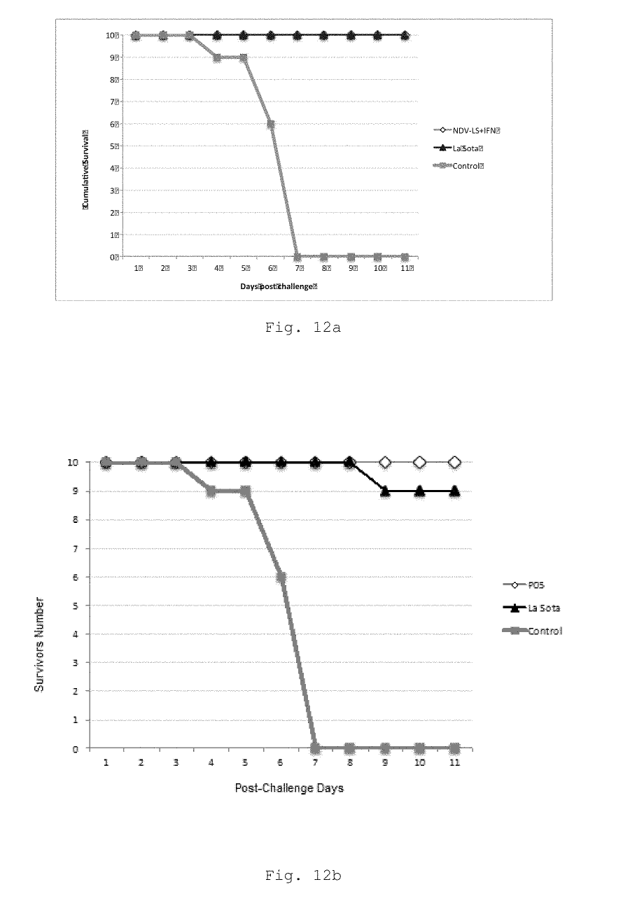

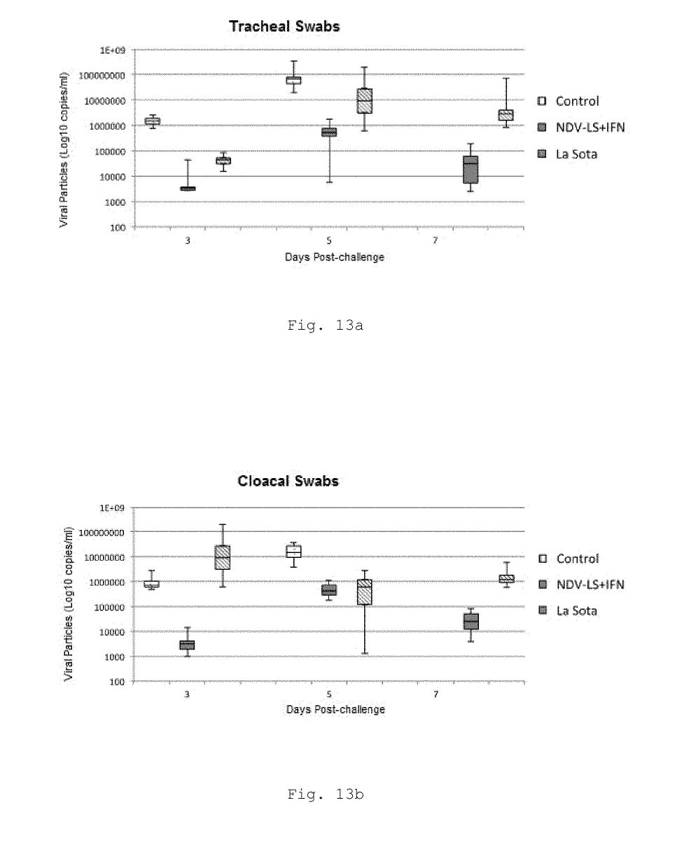

In this example the inventors show the ability of the vaccine virus NDV−LS+IFN to generate protection against the challenge with a Newcastle heterologous virus, genotype V, which is highly pathogenic. For such ends, the survival of challenged poultries and the viral excretion were assessed (FIG. 12 and Table 4). This experiment consisted in assessing 3 groups of 10 poultries each. Group A was vaccinated with NDV−LS−IFN virus, Group B was vaccinated with LaSota strain and Group C was not vaccinated.

Both lyophilized vaccines containing titers ranging from 1×10⁷ to 1×10⁸ DIEP/ml, were applied in poultries of several days of age. The poultries were challenged three weeks after immunization. The challenge virus was a Newcastle disease virus velogenic strain belonging to genotype V, with a 10̂6.0 DIEP/mL titer in 30 microliters. The challenge was carried out by ocular route.

FIGS. 12A and 12B show the survival of the poultries to the challenge with a lethal Newcastle disease virus, it was observed that the poultries immunized with any of the vaccines generate protection in 100% of the poultries.

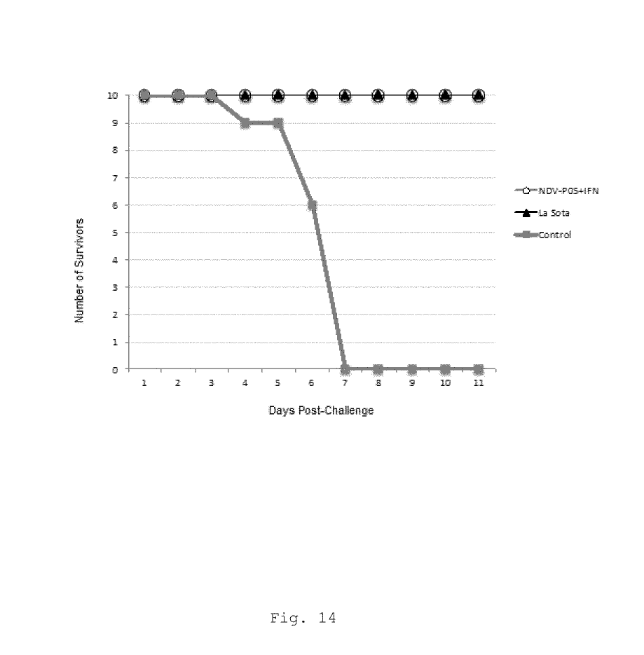

On the other hand, regarding viral excretion, tracheal and cloacal swabs samples were taken at days 3, 5 and 7 post-challenge. Our results indicate that there are fewer poultries excreting the challenge virus when they are vaccinated with NDV+LS+IFN than when they are vaccinated with LaSota virus. This is seen both in the tracheal swabs as in well as in the cloacal swabs (Figures 13A and 13B).

At day 5 post-challenge, it may be seen that 100% of unvaccinated poultries and poultries vaccinated with LaSota virus are excreting the challenge virus. However, only 80% of the poultries vaccinated with NDV−LS+IFN excreted said virus;

i.e., 20% of the challenged poultries are not excreting the challenge virus. Which is an unexpected and very important effect since the advantage of this recombinant virus is to decrease the spread to other poultries.

7 days post-challenge, only 70% of poultries vaccinated with NDV−LS+IFN are excreting the virus through the airway, while 80% of the poultries excrete the challenge virus when they are vaccinated with LaSota virus; i.e., our vaccine decreases by 10% the number of poultries who excrete the virus by the airway. Further, the excretion of the virus via the cloaca is also consistent with previous findings, in this case, 80% of the pouitries vaccinated with. the NDV−LS+IFN virus are still excreting the challenge virus, which is 10% less than the number of vaccinated poultries with LaSota virus, 80%.

Table 4 shows the results of poultries that excreted the challenge virus on the different days of sampling.

TABLE 4
Number of poultries that excreted the challenge virus at days 3, 5 and 7.
The samples were taken from the trachea and the cloaca.

| | Tracheal Swabs | | | Cloacal Swabs | | |
|---|---|---|---|---|---|---|
| | Day 3 | Day 5 | Day 7 | Day 3 | Day 5 | Day 7 |
| NDV-LS + | 10/10 | 8/10 | 7/10 | 10/10 | 8/10 | 8/10 |
| IFN | | | | | | |
| LaSota | 10/10 | 10/10 | 8/10 | 10/10 | 10/10 | 9/10 |
| Control | 10/10 | 8/8 | nr | 10/10 | 8/8 | nr |

nr = not performed

Besides reducing the number of poultries that excrete the challenge virus, the NDV−LS+IFN vaccine also reduces the amount of excreted virus, measured by real time RT-PCR using the oligonucleotides of SEQ ID No. 3 and 4. While both vaccines reduce the viral excretion with respect to the unvaccinated control, the NDV−LS+IFN vaccine further reduces the excretion more than the LaSota vaccine. This is shown in FIG. 13 where it may be seen from day 3 until day 7 post-challenge, both in tracheal samples and in the cloacal samples. This reduction is statistically significant in the samples of day 7 post-challenge.

As it may be seen, unexpectedly, these results show that the poultries vaccinated with the NDV−LS+IFN virus generate a better protection when reducing the number of poultries that excrete the challenge virus and the number of particles released, thus avoiding the spread of the virus.

### EXAMPLE 7

### Assessment of the protection of the NDV−LS+IFN 25 vaccine against the velogenic Newcastle virus.

In this example the inventors show the ability of the vaccine virus NDV−P05+IFN, whose construction is described in Example 2, to generate protection against the challenge of a genotype V Newcastle virus. For such ends, the survival of the challenged poultries and the viral excretion were assessed.

The experimental design consisted in assessing 3 groups of 10 poultries each. Group A was vaccinated with NDV−P05−IFN virus, Group B was vaccinated with LaSota strain and Group C was not vaccinated.

Both lyophilized vaccines containing titers ranging from 1×10⁷ and 1×10⁸ DIEP/ml, were applied in poultries of several days of age. The poultries were challenged three weeks after immunization. The challenge virus was a Newcastle virus velogenic strain, genotype V, with a 10̂6.0 DIEP/mL titer contained in 30 microliters. The challenge was carried out by ocular route.

FIG. 14 shows the survival of the poultries to the challenge with a lethal Newcastle disease virus, said figure shows that the poultries immunized with any of the vaccines generate protection in 100% of the poultries. As it was expected, the unvaccinated birds died between day 3 and day 6 post-challenge.

With respect to the viral excretion, the excretion of the challenge virus was evaluated at days 3, 5 and 7 post-challenge in the 3 assessed groups. The control poultries were monitored at days 3 and 5 post-challenge as the 100% of mortality occurred. before day 7.

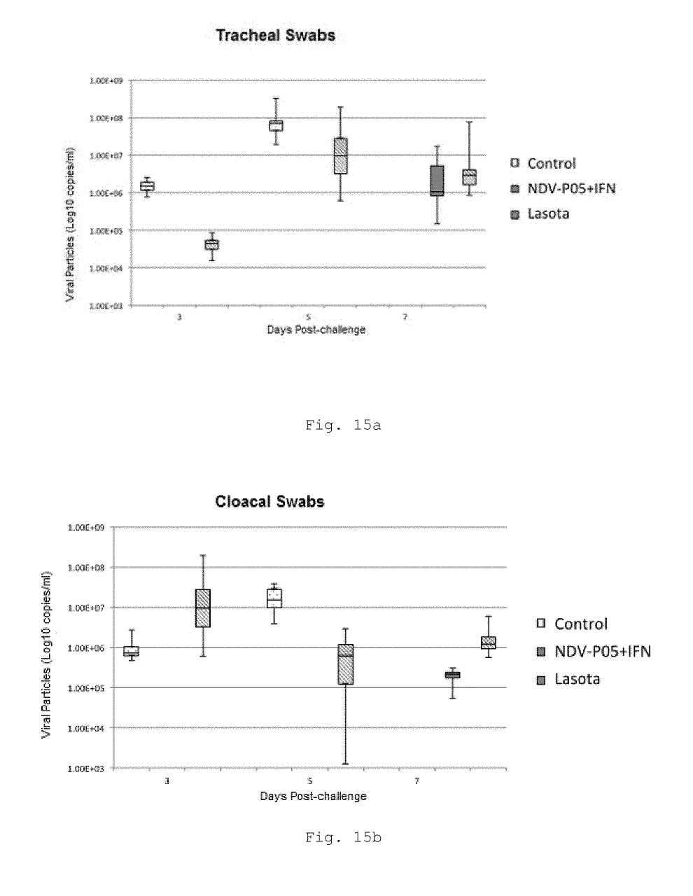

In the poultries vaccinated with the NDV−P05+IFN virus there was no tracheal or cloacal excretion at days 3 and 5 post-challenge, while at day 7 only 4 poultries of 10 are excreting the virus by the tracheal and cloacal routes. Moreover, the poultries vaccinated with LaSota virus are indeed excreting the challenge virus, both tracheal as well as cloacal routes, in the three days of sampling. The poultries vaccinated with the NDV−P05+IFN vaccine excrete less virus than those vaccinated with LaSota virus. This reduction is statistically significant in the cloacal samples of day 7 post-challenge, as it may be seen in FIGS. 15A and 15B.

This result can be partly explained because the vaccine virus NDV−P05−IFN is homologous to the challenge virus; i.e., both are genotype V.

TABLE 5
Number of poultries that excreted the challenge virus at
days 3, 5 and 7. The samples were taken from the trachea and the cloaca.

| | Tracheal Swabs | | | Cloacal Swabs | | |
|---|---|---|---|---|---|---|
| | Day 3 | Day 5 | Day 7 | Day 3 | Day 5 | Day 7 |
| NDV-P05 + | 0/10 | 0/10 | 4/10 | 0/10 | 0/10 | 4/10 |
| IFN | | | | | | |
| LaSota | 10/10 | 10/10 | 8/10 | 10/10 | 10/10 | 9/10 |
| Control | 10/10 | 8/8 | nr | 10/10 | 8/8 | nr |

nr = not performed

Besides reducing the number of poultries that excrete the challenge virus, the NDV−LS+IFN vaccine also reduces the amount of excreted virus, measured by real time RT-PCR using the oligonucleotides of SEQ ID NO. 5 and 6, in the cloacal samples taken at day seven post-challenge, in comparison with the titer obtained by the LaSota Virus (Table 5).

These results as a whole, unexpectedly, demonstrate that the poultries vaccinated with NDV−P05+IFN virus have additional protection than poultries vaccinated with native viruses and also prevents the spread of the challenge virus.

### EXAMPLE 8

### Assessment of the Protection of NDV−LS+IFN Vaccine Against Challenge with High Pathogenicity Avian Influenza Virus H7N3 (H7).

In another example., the NDV−LS+IFN virus whose construction is described in Example 1 was used as seed for the elaboration of a “live” lyophilized vaccine. The lyophilized NDV−LS+IFN virus vaccine was formulated at a final concentration of 2×10⁵ viral particles/dose. This example shows the protection facing a challenge with a high virulence strain of of avian influenza virus subtype H7N3.

The experimental design consisted in assessing 4 groups, each with 10-7 weeks old poultries. One group was vaccinated with NDV−LS+IFN virus, one drop was applied via ocular route. The second group was vaccinated with an emulsified H7N3 30% antigenic mass formulated vaccine, 0.5 ml were applied via subcutaneous route. The third group received a dual vaccination, a drop of NDV−LS+IFN virus and 0.5 ml of 30% of antigenic mass emulsified H7 vaccine were applied via subcutaneous route. The fourth group was not vaccinated and was considered as a control group. Prior to immunization, it was on that the poultries did not have any antibodies against Influenza A H7N3 using the HI technique (Define).

The poultries were observed during 3 weeks after vaccination, at the end of this time a blood sample was taken to assess the presence of antibodies. Subsequently, all the poultries were challenged via ocular route with a drop of a high pathogenicity influenza virus subtype H7N3, which was adjusted to 10-75 DIEP/ml. The birds were observed during two weeks after the challenge in order to determine the survival and the excretion of the challenge virus (Influenza A H7N3) by real time RT-PCR.

### Vaccination-Induced Serologial Response

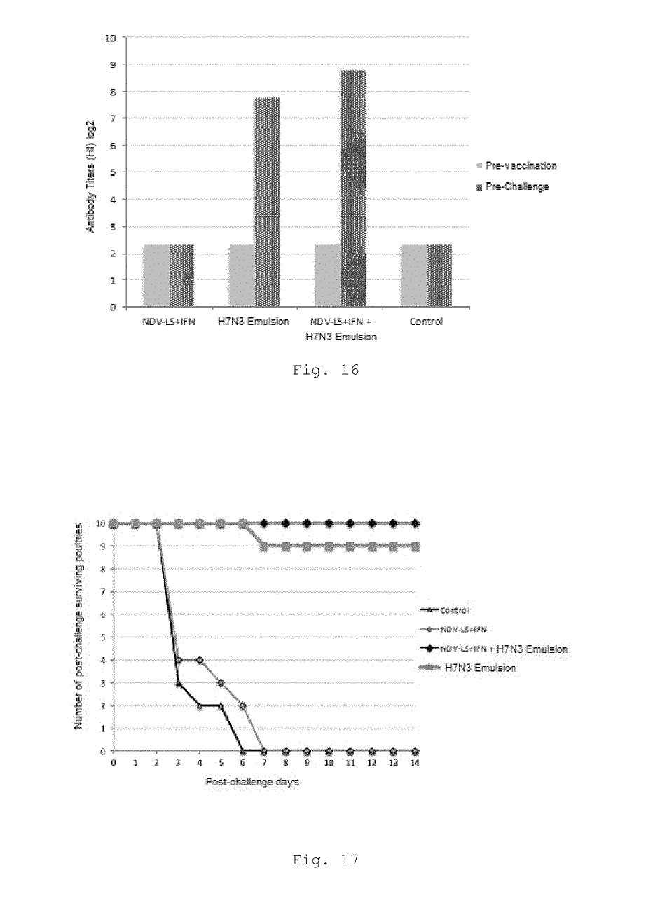

FIG. 16 and Table 6 show the results of poultries serology, both prior to the vaccination and prior to the challenge. The assessment was performed using the HI test of the avian influenza virus H7N3. As it may be seen, only the

Groups 2 and 3, which were immunized with the emulsified H7 vaccine, have an increase in the antibodies titer against H7N3 influenza, while the group that was vaccinated with the live NDV−LS+IFN vaccine maintains the same antibody levels than the control group. While the emulsified H7 vaccine is efficient in the activation of the humoral immune response, this response increased when it was applied together with the live NDV−LS+IFN vaccine (Group 3); i.e., it went from log 2 7.8 to log 2 8.8 (Table 6), which means twice as many antibodies due to dual vaccination. This result indicates that the live vaccine enhances the emulsified vaccine-induced humoral response, very likely due to the biological activity of the interferon.

TABLE 6
Titers values of specific antibodies against the H7N3 avian
influenza virus to vaccination and challenge.

| | | Pre-Vaccination | Pre-Challenge |
|---|---|---|---|
| Group | Description | Titer1 | Titer1 |
| 1 | NDV-LS + IFN | 2.3 | 2.3 |
| 2 | H7N3 Emulsion | | 7.8 |
| 3 | NDV-LS + IFN and H7N3 | | 8.8 |
| | Emulsion | | |
| 4 | Unvaccinated Control | | 2.3 |

Titer in log2 units

The latter is clear in the graph of FIG. 16 and in the data from Table 6, wherein the amount of specific antibodies against the H7N3 virus is twice as high is poultries that were vaccinated with the full H7N3 virus emulsified vaccine and in addition received a second vaccination the same day with the “live” NDVP05+IFN virus with respect to those who only received the live H7N3 virus emulsified vaccine.

On the other hand, it has been reported that one of the functions of the IFN is to activate the T lymphocytes mediated response cooperative through the Th2 pathway. One of the main functions of cooperative lymphocytes (T helper lymphocytes or Th) in the Th2 pathway is the stimulation of the B lymphocytes, who are responsible for the production of specific neutralizing antibodies. These cells (Th lymphocytes) respond to antigenic stimulation and to the signals derived from the APC (antigen presenting cells), proliferating and producing new cytokines, among which are the IL-4, IL-5, IL-6, IL-10 and IL-13 interleukins. Then, the latter also co-stimulate the activation of B lymphocytes for producing antibodies. In addition, some cytokines such as IL-4, IL-5, IL-6, TGFβ and IFNγ itself, further modulate the class or isotype of the antibody produced by the APCs (IL-4: IgG1/IgE; IL-5/IL-4: IgE; TGFβ: IgG2b/IgA: IL-6/IL-4, IgE; IFNγ: IgG3/IgG2a), which promotes the stimilation of the humoral immune response (antibodies) (Binjawadagi et al., 2009; Rajput et at., 2007; Rojas-Espinosa, 2006; Samuel, 2001).

Assessment of the Survival to the Challenge with High Virulence, High Pathogenicity Influenza H7N3 Virus.

The survival results of the poultries are shown in FIG. 17. The best treatment was the dual vaccination (Group 3), NDV−LS+IFN with the H7 Emulsion, which obtained a 100% protection against the challenge. The second place was the H7 Emulsion Group where there the death of a poultry was recorded and therefore we obtained a 90% protection (the poultry died on day 8 after the challenge). The third place was the NDV−LS+IFN vaccine with a 100% mortality after 7 days of the challenge. Finally, the control group had a 100% mortality after 6 days of the challenge. In this experiment, it was possible to appreciate the mortality of the unvaccinated control poultries who die between the 3rd and 6th day post-challenge, while the poultries vaccinated with the NDV−LS+IFN virus die between the 3rd and 7th day, showing a on day delay in the mortality as compared to the control. This shows an interferon effect on the replication of the highly pathogenic H7N3 avian influenza virus.

Furthermore, the difference in 10% protection among the poultries that received the dual vaccination with respect to poultries treated only with emulsified vaccine, can be attributed to the increase in the concentration of antibodies induced by the presence of the NDV−LS+IFN virus, perhaps particularly to the presence of IFN.

These unexpected findings indicate that vaccination with the heterologous NDV−LS+IFN virus in combination with the H7N3 emulsion have the advantage of more efficiently protecting the poultries against infections that are not caused by the Newcastle disease virus, as compared to the application of vaccines for specific diseases such as the avian influenza.

The above suggests that the recombinant viruses of the present invention are effective in the protection of poultries to different diseases, rising the survival of animals racing different types of pathogen agents.

### Viral Excretion

The reduction of the challenge virus excretion is a desirable quality in vaccination. In this example, the quantification of the number of poultries that secrete the challenge virus (H7N3) and the number of viral particles excreted by cloaca and trachea was raised. Samples were taken at days 3, 5 and 7 after the challenge.

Table 7 shows the number of poultries that are excreting the challenge virus both through trachea as well as through cloaca. As you can see, the poultries of the 4 groups excrete the challenge virus, mainly throng the airway (tracheal swabs).

TABLE 7
Excretion of the challenge virus in the cloaca and trachea
of the poultries: Group 1 (NDV-LS + IFN); Group 2 (H7N3 Emulsion);
Group 3 (NDV-LS + IFN + H7N3 Emulsion) and Group 4 (Control).

| | Tracheal Swabs | | | Cloacal Swabs | | |
|---|---|---|---|---|---|---|
| | Day 3 | Day 5 | Day 7 | Day 3 | Day 5 | Day 7 |
| NDV-LS + | 4/4 | 3/3 | 2/2 | 2/4 | 3/3 | 2/2 |
| IFN | | | | | | |
| H7N3 | 10/10 | 10/10 | 8/9 | 2/10 | 10/10 | 9/9 |
| Emulsion | | | | | | |
| NOV-LS + | 9/10 | 8/10 | 10/10 | 0/10 | 4/10 | 9/10 |
| IFN and | | | | | | |
| H7N3 | | | | | | |
| Emulsion | | | | | | |
| Control | 3/3 | 2/2 | nr | 3/3 | 2/2 | nr |

The main differences are seen in the samples of cloacal swabs, where the poultries that are least excreting the virus are those of the group which received a dual vaccination, NDV−LS+IFN and H7 Emulsion (Group 3).

These results were verified by quantifying the titer of the virus by the real time RT-PCR technique. These results, as it was expected, tracheal excretion in the unvaccinated poultries of the control group had the higher excretion titers of the challenge virus, both at days 3 and 5 days post-challenge, there are no data on day 7 because the poultries had already died. The poultries vaccinated with the NDV−LS+IFN vaccine (Group 1) excrete less challenge virus than the unvaccinated poultries. The poultries vaccinated with H7N3 emulsion (Group 2) excrete less virus than the poultries vaccinated with a live NDV−LS+IFN vaccine. Finally, the group of poultries vaccinated with both vaccines (Group 3), NDV−LS+IFN and H7 emulsion, have less excretion titers of challenge virus, both in samples of day 5 and day 7 post-challenge.

This reduction is of approximately 1 logarithm (log 10) when compared with the excretion value obtained with the emulsified H7N3 vaccine and between 2 and 4 logarithms when compared with the excretion of poultries in the control group.

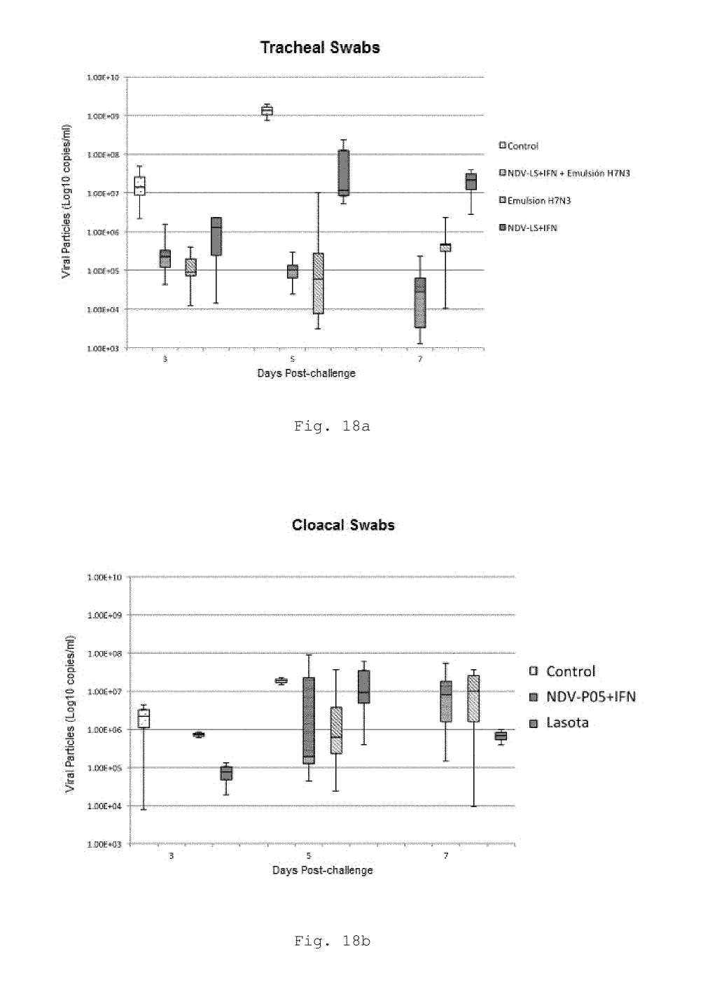

This result indicates a synergistic effect between the two vaccines. These results are shown in FIG. 18A.

On the other hand, the viral excretion measured from cloacal swabs samples had the following behavior; at day three post-challenge, the control poultries excreted more virus than the vaccinated poultries, being the poultries vaccinated with the NDV−LS+IFN vaccine the ones that excrete less virus, titer of 6 vs. 4.6, respectively. At day 5 post-challenge, the viral excretion of the vaccinated poultries is similar to that of the control group, an excretion of between 6.8 and 7.4 is averagely estimated. At day 7 post-challenge, it was seen that the poultries vaccinated with the live vaccine NDV−LS+IFN excreted less viral particles with respect to the other two vaccinated groups; 1.4 and 1.3 logarithms less than the poultries vaccinated with the H7 emulsified vaccine and with the dual vaccination (Group 3), NDV−LS+IFN and H7 emulsion, respectively.

From the cloacal excretion results presented in FIG. 18B, the synergistic effect between the live NDV−LS+IFN vaccine with the H7N3 emulsion was not observed. Surprisingly and unexpectedly, it was observed that the live NDV−LS+IFN vaccine alone further reduces the excretion of the challenge virus than the emulsified vaccine.

Although the interferon antiviral action mechanism is not entirely known, it has been reported that it directly restricts the replication of the viruses at different levels, since its penetration, stripping, RNAm synthesis, protein synthesis and particle assembly. Furthermore, it has been described that the interferon indirectly prevents the viral replication by stimulating, even in the non-infected cells, the synthesis of antiviral proteins. One of these proteins is the 2′5′-oligoadenylate synthetase, an enzyme that synthesizes a 2′5′-oligoadenylate which binds to ribonuclease-L (RNase-L) causing its dimerzation. The activated RNase-L causes massive degradation of both viral and cell mRNAs, thereby inhibiting the synthesis of proteins in the infected cells and the apoptosis thereof, which leads to a remarkable antiviral capacity. Other proteins such as the PKR (double stranded RNA-dependent protein kinase), whose levels are increased by the effect of the viral infection, interfere with the replication of the viral genome with any new infecting virus Samuel 2001; Rojas-Espinoza, 2006; Binjawadagi, et. al., 2009).

Once again it is shown that the heterologous viruses of the present invention are effective in reducing the excretion time of the viruses used in the challenge with pathogens that are different to the Newcastle disease. This advantage is extremely important because it reduces the likelihood of infection in these animals even from diseases other than the Newcastle disease. Likewise, the heterologous virus vaccines of the present invention may be used to protect poultries not only from the Newcastle disease, but from other diseases with different etiological agents such as the avian influenza.

All the publications and patents herein mentioned are incorporated in the present invention as reference. Several modifications and variations to the vaccines, viruses and methods described in the present invention may be appreciated by those skilled in the art and without departing from the scope and spirit of the present invention. Although the present invention has been described with respect to several specific embodiments, it shall be understood that the present invention as claimed shall not be undue limited to such embodiments. In fact, several modifications of the described modes for carrying out the invention, which are evident to those skilled in the art and in the related fields, are intended to be within the scope of the following claims.

### REFERENCES

Absalon, A. E., Mariano-Matias, A., Vasquez-Marquez, A., Morales-Garzon A., Cortes-Espinosa, D. V., Ortega-Garcia, R., Lucio-Decanini, E., 2012. Complete genome sequence of a velogenic Newcastle disease virus isolated in Mexico. Virus genes 45, 304-310.
Afonso, C., Miller, P., 2013. Newcastle disease: progress and gaps in the development of vaccines and diagnostic tools. Vaccines and Diagnostics for Transboundary Animal Diseases, 94.
Aldous, E., Alexander, D., 2008. Newcastle disease in pheasants (Phasianus colchicus): a review. The Veterinary Journal 175, 181-185.
Aldous, E., Mynn, J., Banks, J., Alexander, D., 2003. A molecular epidemiological study of avian paramyxovirus type 1 (Newcastle disease virus) isolates by phylogenetic analysis of a partial nucleotide sequence of the fusion protein gene. Avian pathology 32, 239-257.
Aldous, E. W. Alexander, D. J., 2001. Detection and differentiation of Newcastle disease virus (avian paramyxovirus type. Avian pathology 30, 117-128.
Alexander, D., Manvell, Lowings, J., Frost, K., Collins, M., Russell, P., Smith, J., 1997. Antigenic diversity and similarities detected in avian paramyxovirus type 1 (Newcastle disease virus) isolates using monoclonal antibodies. Avian pathology 26, 399-418.
Alexander, D. J., Aldous, E. W., Fuller, C. M., 2012. The long view: a selective review of 40 years of Newcastle disease research. Avian pathology: journal of the W.V.P.A 41, 329-335.
Alexander, D. J., Bell, J. G., Alders, R. G., 2004. A technology review: Newcastle disease, with special emphasis on its effect on village chickens. Food & Agriculture Org.
Alvarado, A. I. Tenorio., A. S. Aguilar., Sandoval, A. H., 2006. Promesas y limitaciones de las vacunas génicas. Rev. Mex. De ciencias farmacéuticas. 37, 26-37.
Ballagi-Pordány, A., Wehmann, E., Herczeg, J., Belák, S., Lomniczi, B., 1996. Identification and grouping of Newcastle disease virus strains by restriction site analysis of a region from the F gene. Arch Virol 141, 243-261.
Bidin, Z., Bidin, Z., 2008. Diseases of poultry. Veterinarski Fakultet Sveučilišta u Zagrebu (Faculty of Veterinary Medicine, University of Zagreb).
Binjawadagi, B., Babu, Y. H., Sreekumar, E., 2009. Immunomodulatory effect of recombinant chicken interferon-gamma (rchIFN-) on specific and non-specific immune responses in chicken vaccinated against Newcastle Disease Virus (NDV). International Journal of Poultry Science 8, 122-127.
Borland, L., Allan, W., 1980. Laboratory tests for comparing live lentogenic Newcastle disease vaccines. Avian pathology 9, 45-59.
Cantin, C., Holguera, J., Ferreira, L., Villar, E., Muñoz-Barroso, I., 2007. Newcastle disease virus may enter cells by caveolae-mediated endocytosis. J Gen Viral 88, 559-569.
Carter, G. R., Wise, D. J., Flores, E. F., 2004. A Concise Review of Veterinary Virology. IVIS.
Cornax, I., Miller, P. J., Afonso, C. L., 2012. Characterization of live LaSota vaccine strain-induced protection in chickens upon early challenge with a virulent Newcastle disease virus of heterologous genotype. Avian diseases 56, 464-470.
Curran, J., Marq, J.-B., Kolakofsky, D., 1995. An N-terminal domain of Sendai paramyxovirus P protein acts as a chaperone for the NP protein during the nascent chain assembly step of genome replication. J Viral 69, 849-855.
Czeglédi, Ujvári, D., Somogyi, E., Wehmann, E., Werner, O., Lomniczi, B., 2006. Third genome size category of avian paramyxovirus serotype 1 (Newcastle disease virus) and evolutionary implications. Virus Research 120, 36-48.
Diel, D. G., da Silva, L. H. A., Liu, H., Wang, Z., Miller, P. J., Afonso, C. L., 2012. Genetic diversity of avian paramyxovirus type 1: Proposal for a unified nomenclature and classification system of Newcastle disease virus genotypes. Infection, Genetics and Evolution 12, 1770-1779.
Dortmans, J., 2011. Virulence Determinants of Newcastle Disease virus.
Dortmans, J., Rottier, P., Koch, G., Peeters, B., 2010. The viral replication complex is associated with the virulence of Newcastle disease virus. J Viral 84, 10113-10120.
Dortmans, J. C., Koch, G., Rattier, P. J., Peeters, B. P., 2011. Virulence of newcastle disease virus: what is known so far? Veterinary Research 42, 1-11.
Ecco, R., Brown, C., Susta, L., Cagle, C., Cornax, I., Pantin-Jackwood, M., Miller, P. J., Afonso, C. L., 2011. In vivo transcriptional cytokine responses and association with clinical and pathological outcomes in chickens infected with different Newcastle disease virus isolates using for paraffin-embedded samples. Veterinary immunology and immunopathology 141, 221-229.
Egelman, E., Wu, S., Amrein, M., Partner, A., Murti, G., 1989. The Sendai virus nucleocapsid exists in at least four different helical states. J Viral 63, 2233-2243.
Engel-Herbert, I., Werner, O., Teifke, J. P., Mebatsion, T. Mettenleiter, T. C., Römer-Oberdörfer, A., 2003. Characterization of a recombinant Newcastle disease virus expressing the green fluorescent protein. Journal of virological methods 108, 19-28.
Garten, W. Perk, W., Nagai Y., Rott, R., Klenk, H.-D., 1980. Mutational changes of the protease susceptibility of glycoprotein F of Newcastle disease virus: effects on pathogenicity. J Gen Virol 50, 135-147.
Hines, N. L. Miller, C. L., 2012. Avian paramyxovirus serotype-1: a review of disease distribution, clinical symptoms, and laboratory diagnostics. Veterinary medicine international 2012.
Hironori, S., Seisuke, H., Nobuhiro, I., Yasutada, I., Masao, K., 1987. Nucleotide sequence of the hemagglutinin-neuraminidase gene of Newcastle disease virus avirulent strain D26: evidence for a longer coding region with a carboxyl terminal extension as compared to virulent strains. Virus Research 8, 217-232.
Hu, S., Ma, H., Wu, Y., Liu, W., Wang, X., Liu, Y., Liu, X., 2009a. A vaccine candidate of attenuated genotype VII Newcastle disease virus generated by reverse genetics. Vaccine 27, 904-910.
Hu, S., Ma, H., Wu, Y., Liu, W., Wang, X., Liu, Y., Liu, X., 2009b. A vaccine candidate of attenuated genotype VII Newcastle disease virus generated by reverse genetics. Vaccine 27, 904-910.
Hu, Z., Hu, S., Meng, C., Wang, X., Zhu, J., Liu, X., 2011. Generation of a Genotype VII Newcastle Disease Virus Vaccine Candidate with High Yield in Embryonated Chicken Eggs. Avian diseases 55, 391-397.
Huang, Z., Panda, A., Elankumaran, S., Govindarajan, D., Rockemann, D. D., Samal, S. K., 2004. The hemagglutinin-neuraminidase protein of Newcastle disease virus determines tropism and virulence. J Viral 78, 4176-4184.
IASA—Investigación Aplicada S.A. de C.V., I.I.A., S.A., de,
C.V., 2012., Genovax, N5., Retrieved, from, <http://www.engormix.com/iasa-investigacion-aplicada/genovax-, sh10043_pr26921.htm>.
Kaiser, P., 2010. Advances in avian immunology—prospects for disease control: a review. Avian pathology: journal of the W.V.P.A 39, 309-324.
Kapczynski, D. R., Afonso, C. L., Miller, P. J., 2013. Immune responses of poultry to Newcastle disease virus. Developmental & Comparative Immunology 41, 447-453.
Kapczynski, D. R., King, D. J., 2005. Protection of chickens against overt clinical disease and determination of viral shedding following vaccination with commercially available Newcastle disease virus vaccines upon challenge with highly virulent virus from the California 2002 exotic Newcastle disease outbreak. Vaccine 23, 3424-3433.
Karaca, K., Sharma, J. M., Winslow, B. J., Junker, D. E., Reddy, S., Cochran, M., McMillen, J., 1998. Recombinant fowlpox viruses coexpressing chicken type I IFN and Newcastle disease virus HN and F genes: influence of IFN on protective efficacy and humoral responses of chickens following in ovo or post-hatch administration of recombinant viruses. Vaccine 16, 1496-1503.
Kim, L. M., King, D. J., Curry, P. E., Suarez, D. L., Swayne, D. E., Stallknecht, D. E., Slemons, R. D., Pedersen, J. C., Senne, D. A., Winker, K., 2007. Phylogenetic diversity among low-virulence Newcastle disease viruses from waterfowl and shorebirds and comparison of genotype distributions to those of poultry-origin isolates. J Viral 81, 12641-12653.
Kim, S.-H., Samal, S. K., 2010. Role of Untranslated Regions in Regulation of Gene Expression, Replication, and Pathogenicity of Newcastle Disease Virus Expressing Green Fluorescent Protein. J Viral 84, 2629-2634.
King, A. M., Adam, M. J., Lefkowitz, E. J., 2012. Virus taxonomy: ninth report of the International Committee on Taxonomy of Viruses. Elsevier.
Knipe, D. M., 2013. Fields Virology. Wolters Kluwer Health. Krishnamurthy, S., Samal, S. K., 1998. Nucleotide sequences of the trailer, nucleocapsid protein gene and intergenic regions of Newcastle disease virus strain Beaudette C and completion of the entire genome sequence. J Gen Viral 79 (Pt 10), 2419-2424.
Lamb, R. A., Paterson, R. G., Jardetzky, T. S., 2006. Paramyxovirus membrane fusion: lessons from the F and HN atomic structures. Virology 344, 30-37.
Lewis, P. J., Cox, G. J., Babiuk, L. A., 1997. Polynucleotide vaccines in animals: enhancing and modulating responses. Vaccine 15, 861-864.
Li, J., Melanson, V. R., Mirza, A. M., Iorio, R. M., 2005. Decreased dependence on receptor recognition for the fusion promotion activity of L289A-mutated Newcastle disease virus fusion protein correlates with a monoclonal antibody-detected conformational change. J Virol 79, 1180-1190.
Locke, D. P., Sellers, H. S., Crawford, J. M., Schultz-Cherry, S., King, D. J., Meinersmann, R. J., Seal, B. S., 2000. Newcastle disease virus phosphoprotein gene analysis and transcriptional editing in avian cells. Virus Research 69, 55-68.
Lopez, H. S., Olvera, L. G., 2010. Farmacologia clinica en ayes comerciales. McGraw-Hill Interamericana.
Manrique, C. E. M., 2005. Modulación de la respuesta inmune. Tendencias vigentes. Medisan 9, 3.
Mayo, M. A., 2002. A summary of taxonomic changes recently approved by ICTV. Arch Viral 147, 1655-1656.
McFerran, J. McCracken, R., 1988. Newcastle disease, Newcastle disease. Springer, pp. 161-183.
Mebatsion, T., de Vaan, L. T., de Haas, N., Römer-Oberdörfer, A., Braber, M., 2003. Identification of a mutation in editing of defective Newcastle disease virus recombinants that modulates P-gene mRNA editing and restores virus replication and pathogenicity in chicken embryos. J Viral 77, 929-9265.
Mebatsion, T., Koolen, M. J., de Vaan, L. T., de Haas, N., Braber, M., Römer-Oberdörfer, A., van den Elzen, P., van der Marel, P., 2002. New castle disease virus (NDV) marker vaccine: an immunodominant epitope on the nucleoprotein gene of NDV can be deleted or replaced by a foreign epitope. J Viral 76, 10138-10146.
Mebatsion, T., Verstegen, S., De Römer-Oberdörfer, A., Schrier, C. C., 2001. A recombinant Newcastle disease virus with low-level P protein expression is immunogenic and lacks pathogenicity for chicken embryos. J Virol 75, 420-428.
Miller, P. J., Afonso, C. L., El Attrache, J., Dorsey, K. M., Courtney, S. C., Guo, Z. Kapczynski, D. R., 2013. Effects of Newcastle disease virus vaccine antibodies on the shedding and transmission of challenge viruses. Developmental & Comparative Immunology 41, 505-513.
Miller, P. J., Decanini, E. L. Afonso, C. L., 2010. Newcastle disease: evolution of genotypes and the related diagnostic challenges. Infection, genetics and evolution journal of molecular epidemiology and evolutionary genetics in infectious diseases 10, 26-35.
Miller, P. J., Estevez, C., Yu, Q., Suarez, D. L., King, D. J., 2009. Comparison of viral shedding following vaccination with inactivated and live Newcastle disease vaccines formulated with wild-type and recombinant viruses. Avian diseases 53, 39-49.
Miller, P. J., King, D. J., Afonso, C. L., Suarez, D. L., 2007. Antigenic differences among Newcastle disease virus strains of different genotypes used in vaccine formulation affect viral shedding after a virulent challenge. Vaccine 25, 7238-7246.
Murphy, F. A., Fauquet, C. M., Bishop, D. H., Ghabrial, S. A., Jarvis, A. W., Martelli, G. P., Mayo, M. A., Summers, M. D., 1995. Virus taxonomy: classification and nomenclature of viruses. Sixth report of the International Committee on Taxonomy of Viruses. Springer-Verlag.
Murphy, F. A., Gibbs, E. P. J., Horzinek, M. C., Studdert, M. J., 1999. Veterinary Virology. Elsevier Science.
Nakaya, T., Cros, J., Park, M.-S., Nakaya, Y., Meng, H., Sagrera, A., Villar, E., Garciá-Sastre, A., Palese, P., 2001. Recombinant Newcastle disease virus as a vaccine vector. J Viral 75, 11868-11873.
Peeters, B. P., de Leeuw, O. S., Koch, G., Gielkens, A. L., 1999. Rescue of Newcastle disease virus from cloned cDNA: evidence that cleavability of the fusion protein is a major determinant for virulence. J Virol 73, 5001-5009.
Perozo F., Villegas, P., Estevez, C., Alvarado, I., Purvis, L. B., 2006. Use of FTA filter paper for the molecular detection of Newcastle disease virus. Avian pathology: journal or the W.V.P.A 35, 93-98.
Poch, O., Blumberg, B. M. Bougueleret, L., Tordo N., 1990. Sequence comparison of five polymerases (L proteins) of unsegmented negative-strand RNA viruses: theoretical assignment of functional domains. J Gen Virol 71, 1153-1162.
Rajput, Z. I., Hu, S.-h., Xiao, C. A., Arilo, A. G., 2007. Adjuvant effects of saponins animal immune responses. Journal of Zhejiang University Science B 8, 153-161.
Rauw, F., Gardin, Y., Palya, V., van Borm, S., Gonze, M., Lemaire, S., Van den Berg, T., Lambrecht, B., 2009. Humoral, cell-mediated and mucosal immunity induced by oculo-nasal vaccination of one-day-old SPF and conventional layer chicks with two different live Newcastle disease vaccines. Vaccine 27, 3631-3642.
Rojas-Espinosa, Ó., 2006. Immunología (de memoria). Editorial Medica Panamericana S.A. de C.V.
Römer-Oberdörfer, A., Mundt, E., Mebatsion, T., Buchholz, U. J., Mettenleiter, T. C., 1999. Generation of recombinant lentogenic Newcastle disease virus from cDNA. J Gen Virol 80, 2987-2995.
Römer-Oberdörfer, A., Werner, O., Veits, J., Mebatsion, I. Mettenleiter, T. C., 2003. Contribution of the length of the HN protein and the sequence of the F protein cleavage site to
Newcastle disease virus pathogenicity. J. Gen. Viral 84, 3121-3129.
Rue, C. A., Susta, L., Cornax, Brown, C. C., Kapczynski, D. R., Suarez, D. L., King, D. J., Miller, P. J., Afonso, C. L., 2011. Virulent Newcastle disease virus elicits a strong innate immune response in chickens. J Gen. Viral 92, 931-939.
Russell, P. H., Ezeifeka, G. O., 1995. The Hitchner B1 strain of Newcastle disease virus induces high levels of IgA, IgG and IgM in newly hatched chicks. Vaccine 13, 61-66.
Samuel, C. E., 2001. Antiviral actions of interferons. Clinical microbiology reviews 14, 778-809, table of contents.
San Roman, K., Villar, E., Munoz-Barroso, I., 1999. Acidic pH enhancement of the fusion of Newcastle disease virus with cultured cells. Virology 260, 329-341.
Sawant, P. M., Verma, P. C., Subudhi, P. K., Chaturvedi, U., Singh, M., Kumar, R., Tiwari, A. K., 2011. Immunomodulation of bivalent Newcastle disease DNA vaccine induced immune response by co-delivery of chicken IFN-γ and IL-4 genes. Veterinary immunology and immunopathology 144, 36-44.
Seal, B. S., King, D. J., Sellers, H. S., 2000. The avian response to Newcastle disease virus. Developmental Comparative Immunology 24, 257-268.
Steward, M., Vipond, I. B., Emmerson, P. T., 1993. RNA editing in Newcastle disease virus. J Gen Viral 74, 2539-2548.
Wegenka, U. M., Lütticken, C., Buschmann, J., Yuan, J.,
Lottspeich, F., Müller -Esterl, W., Schindler, C., Roeb, E., Heinrich, P. C., Horn, F., 1994. The interleukin-6-activated acute-phase response factor is antigenically and functionally related to members of the signal transducer and activator of transcription. (STAT) family. Molecular and Cellular Biology 14, 3186-3196.
White, J. M., Delos, S. E., Brecher, M., Schornberg, K., 2008. Structures and mechanisms of viral membrane fusion proteins: multiple variations on a common theme. Critical reviews in biochemistry and molecular biology 43, 189-219.
Wigley, P., Kaiser, P., 2003. Avian cytokines in health and disease. Revista Brasileira de Ciência Avícola 5, 1-14.
Wise, M. G., Sellers, H. S., Alvarez, R., Seal, B. S., 2004. RNA-dependent RNA polymerase gene analysis of worldwide Newcastle disease virus isolates representing different virulence types and their phylogenetic relationship with other members of the paramyxoviridae. Virus Research 104, 71-80.
Yan, Y., Samal, S. K., 2008. Role of intergenic sequences in
Newcastle disease virus RNA transcription and pathogenesis. J Viral 82, 1323-1331.
Zhao, W., Spatz, S., Zhang, Z., Wen, G., Garcia, M., Zsak, L., Yu, Q., 2014. Newcastle disease virus (NDV) recombinants expressing infectious larynqotracheitis virus (ILTV) glycoproteins gB and gD protect chickens against ILTV and NDV challenges. J Viral 88, 8397-8406.

1. A recombinant Newcastle Disease virus comprising at least one heterologous foreign sequence.

2. The recombinant Newcastle Disease virus according to claim 1, wherein the heterologous foreign sequence is derived from genes encoding for cytokines of the genus Gallus gallus.

3. The recombinant Newcastle Disease virus according to claim 2, wherein the cytokine of the genus Gallus gallus may be Interleukin-6, Interferon gamma, among others.

4. The recombinant Newcastle Disease virus according to claim 2, wherein the recombinant virus may express the proteins, Interferon gamma, Interleukin-6, among others, of the genus Gallus gallus.

5. The recombinant Newcastle Disease virus according to claim 1, wherein the recombinant virus comes from the Newcastle Disease virus (NDV), LaSota pNDVLS strain or NDV−P05 strain.

6. The recombinant Newcastle Disease virus according to claim 1, wherein the recombinant Newcastle Disease virus that comes from the NDV, LaSota strain, has the SEQ ID NO. 2 (NDV−LS−IFN).

7. The recombinant Newcastle Disease virus according to claim 1, wherein the recombinant Newcastle Disease virus that comes from the NDV, NDV−PO5 strain, has the SEQ ID NO. 1 (NDV−P05−IFN).

8. A method for producing a recombinant Newcastle Disease virus according to claims 6 and 7, comprising the steps of:
a) Infecting a cell with the NDV virus
b) Transfecting the infected cell with a first vector construct comprising an heterologous gene for the NDV genome, and an NDV sequence capable of targeting the integration of the heterologous gene to an insertion site of the NDV genome.
c) identifying, isolating and optionally purifying the generated recombinant NDV;
d) Repeat the previous steps using the recombinant NDV virus obtained from the previous steps to infect the cell and an additional vector construct comprising and additional gene which heterologous for the NDV genome and homologous for the gene of the first vector construct.

9. The use of the recombinant Newcastle Disease virus according to any of claims 1 to 7, to prepare a veterinary pharmaceutical composition for modulating the immune response against avian diseases.

10. A veterinary pharmaceutical composition to induce an immune response in the host against avian diseases, comprising a recombinant Newcastle Disease virus according to any of claims 1 to 7, a pharmaceutically acceptable carrier, diluent, adjuvant and/or additive.

11. The use of the composition according to claim 10, wherein the immune response is an increase to the immunomodulator proteins expression in poultries.

12. The use according to claim 12, wherein the immunomodulator protein may be Interferon gamma or Interleukin-6.

13. A vaccine comprising a recombinant Newcastle Disease virus according to any of claims 1 to 7, a pharmaceutically acceptable carrier, diluent, adjuvant and/or additive.

14. The use of the vaccine according to claim 13, to induce an immune response in the host against avian diseases, wherein said immune response is an increase in avian cytokines.

15. The use according to claim 14, wherein the increase in cytokines is an increase in Interferon gamma or interleukin-6.

16. The use according to claim 15, wherein the increase in the Interferon-gamma or Interleukin-6 expression is maintained during at least 6 weeks after vaccination.

17. The use of the vaccine according to claim 13, wherein said vaccine reduces the viral spread due to a decrease in the viral excretion of the pathogen agent in a shorter time as compared with other vaccine containing native Newcastle Disease viruses.

18. A formulation containing a combination of vaccines, wherein said formulation is comprised by the vaccine according to claim 13 and other avian vaccine such as that of H7N3 influenza and a pharmaceutically acceptable carrier, diluent, adjuvant and/or additive.

19. The use of the formulation according to claim 18 to protect poultries from viral infections, wherein said formulation increases the protection of the poultries by elevating the levels of survival of poultries as compared to mono-vaccine formulations.

20. The use of the formulation according to claim 19 to protect poultries from viral infections, wherein the protection is increased because said formulation increases the levels of antibodies that result from the elevation of interferon gamma induced by the recombinant viruses of claims 1 to 7.

21. Oligonucleotides to identify the heterologous Newcastle Disease virus, LaSota strain, of SEQ ID NO. 2 (NDV−LS−IFN) wherein said oligonucleotides have the SEQ ID NO. 3 and SEQ ID NO. 4 and selectively amplify a DNA sequence containing the heterologous gene.

22. Oligonucleotides to identify the heterologous Newcastle Disease virus, P05 strain, of SEQ ID NO. 1 (NDV−P05−IFN) wherein said oligonucleotides have the SEQ ID NO. 5 and SEQ ID NO. 6 and selectively amplify a DNA sequence containing the heterologous gene.

23. An in vitro or ex vivo method to identify the heterologous recombinant NDV viruses according to claims 6 and 7, comprising contacting the virus with the oligonucleotides according to claims 21 or 22, which amplify the flanking sequence containing the insertion sites of the heterologous gene using the Reverse Transcription Polymerase Chain Reaction


SEQUENCE LISTING (6 sequences)

SEQ ID NO: 1 - DNA, 15786 bp, Newcastle Disease Virus
accaaacagagaatcggtgagttacgataaaaggcgaagaagcaatcgaaatcgtacgggtagaaggtgtgaacctcgagtgcgaggccgaaactcaaactcgaggaagccttctaccgacatgtcgtccgtatttgacgaatacgagcaactcctcgctgctcagactcgccctaatggagctcacggaggaggagagaaagggagcactttaaaagttgaggtcccagtatttactcttaatagtgacgatccagaagatagatggaatttttcagtattctgtcttcggattgccgttagtgaggatgccaacaagccactcaggcaaggtgctcttatatccctcttatgctcccattctcaagtgatgaggaaccatgttgcccttgcagggaaacagaatgaggccacactggctattcttgagattgatgggtttaccaacagcacgccccagttcaacaacaggagtggagtgtccgaggagagagcacagagattcatggtgatagccgggtctctccctcgggcatgcagcaacggtactccgttcgtcacagccggggttgaagatgatgcaccagaagacatcactgacactctggaaaggatcctatctatccaggctcaggtatgggtcacagtagcgaaggccatgactgcatatgagacagcagatgagtcggaaacaagaagaatcaataagtatatgcagcaaggcagagtccaaaagaaatacatcctccaccctgtatgcaggagtgcaattcaactcacaatcaggcattctttggcagttcgcattttcttagttagcgagcttaagagaggccgcaatacggcaggtgggagctccacatattacaacttagtaggggatgtagactcatacatcaggaacaccgggcttactgcattcttcttgacactcaaatatggaattaataccaagacgtcagctcttgcactcagcagcctcacaggcgatatccaaaaaatgaagcagctcatgcgtttatatcggatgaagggagaaaatgcaccgtacatgacattgctaggcgacagtgaccagatgagctttgcaccagctgagtatgcacaactttattcttttgccatgggcatggcatcagtcttagataaaggaactggcaaataccaattcgccagagactttatgagcacatcattctggagacttggggtagaatatgctcaggctcaggggagtagcatcaatgaggacatggctgctgagctaaaactaaccccggcagcaaggagaggcctggcagctgctgcccaacgagtgtctgaggaaattggcagcatggatattcctactcaacaggccggggtcctcactggactcagcgacggaggcccccaagccccacagggcggatcgaacaagtcgcaagggcaaccggatgccggggacggagagacccaattcttggatttgatgagagcggtggcgaatagcatgcgagaagcacaaaattccgcaccgagcaccacccatccagagcctcccccaactcctgggccatccccagacaacgacaccgactgggggtactgattgacaacacccagcctgcccccacagggtcgccccaaatcctccgcccgaacccctccctcactccccgacccacagcctcgcatggccgagccaacgaaagtacccccccatcctctctcccacccccagctgcacaaccccacccgcccaaagcaacacaggcgcaacttgatccaccagcagtccatgcagagccaaagatattagaaaaaaatacgggtagaagagagacgcccagagaccaggacaagtcaccaaggtctctgttctcccttctacccagtggactagggcgaagatggctacctttacagatgcggagatagacgatatacttgagaccagtggaactgtcattgacagcataatcacggcccagggtaaatcagcagagaccctcggaaggagcgcaatcccgcaaggcaagaccaaagcaccgagcacagcatgggagaaacatggggacatccaaccatccgccagtcaggacacccctgaccaacaggatggatcagacaaacagccatccacacccaagcaggcgacctcacacaacagcccgccagccacacccaccgaaccgccccccactcaggccgcgggcgaggctggcgacgcgcagctcaagaccggagcaagcaactctcttttgtccatgctcgacaagctgagcaataaatcgtctaatactaaaaagggcccatggtcaagtcctcaagaaggacatcaccaacctccggccaaacaacatgggaatcagccgagccacgggaaccaccaggaaaaatctcagcaccaggccaaggccgcccctggaagccggggcacagacgtgaacacagcatatcatggacaacagaaggagtcacaactatcagctggtgcaacccctcatgcgctccagtcagggcagagccaagacaatactcctgtatctgtggatcatgtccagccacctgtcgactttgtgcaggcaatgatgtctacgatggaggcgttattgcagaaggtaaataaagttgactatcagctagatctagtcttgaagcagacatcctccatccctatgatgcgaactgaaatccaacagctgaaaacatctgttgcggtcatggaagccaatttaggcatgatgaaaattctggaccctggttgtgctaacatttcatccttaagcgatctacgggcagtcgctcgatcccacccagttttagtttcaggccccggagatccgtctccttatgtgacacaaggaggtgaattgacactcaataaactctcgcaaccggtacaacacccttccgagttaattaaatccgccgcggcaagcgggcctgacatgggagtggagaaagacactgtccgtgcattgatcacttcacgcccgatgcatccgagctcctcagctaagctcctgagtaagctggatgcagccaggtcgattgaggagatcaggaagatcaaacgccttgcgctgaatggttgatcaccatcacaacctacaacaggttcctgcctctttagtgtcacaagggatccgtcctaggcccctccccacaagtctacgctccagcactccaagtaacaaccctcgaaattgacgcgatcgatgcaaccgcggattaagaaaaaatacgggtagaagatcgaagcgctgatccttctgccaccatggcttgccagacttacaacttgtttgttctgtctgtcatcatgatttattatggacatactgcaagtagtctaaatcttgttcaacttcaagatgatatagacaaactgaaagctgactttaactcaagtcattcagatgtagctgacggtggacctattattgtagagaaactgaagaactggacagagagaaatgagaaaaggatcatactgagccagattgtttcgatgtacttggaaatgcttgaaaacactgacaagtcaaagccgcacatcaaacacatatctgaggagctctatactctgaaaaacaaccttcctgatggcgtgaagaaggtgaaagatatcatggacctggccaagctcccgatgaacgacttgagaatccagcgcaaagccgcgaatgaactcttcagcatcttacagaagctggtggatcctccgagtttcaaaaggaaaaggagccagtctcagaggagatgcaattgctaatgggtttaaactggatccatttcgaacctcctctaccccattgaatgatcgcgcaactacagttaatctaacagcattgaagattaagaaaaaatacgggtagaatcaaagtgccctgactgtgccaaaatggactcatccaggacaatcggactgtactttgattctgcccttccttccagcagcctgcttgcttttccgattgtccttcaaaccacaggagatgggaagaagcaaatcaccccacaatataggatccaacggcttgacttgtggacggacagcaaagaagattcggtttttatcaccacatacgggttcatttttcaagtcggaaatgaagaagtcaccgttggcacgattaacgataaccccaggcaagagttactttcttctgcaatgctttgccttgggagtgtccctaatgacggagatctcgttgagctggcgagggcctgcctcactatggtggtaacgtgcaagaagagtgcaaccaatactgagagaatagttttttcgatagtgcaggcaccccgggtgctgcaaagctgtatggttgtggcaaataggtactcatcagtgaatgcagttaagcatgtaaaagcccccgagaagattcccgggagcggaactttagagtataaggtgaactttgtctctttgactgtggtgccgaggaaggatgtctacaggatcccaaccgcagcattgaaagtatctggctcgagcctgtataatcttgcgctcaatgtcactattgatgtggaggtggatccgaagagcccattagtcaaatccctttccaagtccgatagtggatactatgctaaccttttcttacatatcgggcttatgtccactgtagataaaaagggaaagaaagtgacatttgacaagctggaaaggaagataagaagactcaatctatctgttgggctcagtgatgtgctcggaccttccgtgcttgtgaaggcgagaggtgcacggactaagctgttggcacctttcttctctagcagtgggacagcctgctaccctatagcaaatgcttctccccaggttgctaagatactctggagtcagactgcgcacctgcgaagtgtaaaggtcatcattcaagcaggcacccaacgtgctgtcgcagtgaccgctgatcatgaggttacctctactaagatagagaagaggcatactattgctaaatacaacccttttaagaaataggttgtattcctgatactatggtctgcccgctttcttgaatcattgcgacactagataatgatctgctttgattgcttatagttagtttacctgtctatccaattagaaaaaacacgggtagaagagtccggatcccggccggcacatccaaggcgcagtatgggctccagaccttctaccaagaactcagtacctctgatgctgatcacccggattacgctgatactgagctgtatctgtctgacaagctctcttgacggcagacctcttgcagctgcagggattgtggtaacgggagacaaagcagtcaatatatacacttcatctcaaacagggtcgatcatagtcaagttactccctaacatgcccaaagataaagaggcgtgcgcaaaagccccattggaggcatacaatagaacactgaccactttactcacccctcttggtgactccatccggaggatacaagggtctgcgactacatctggaggggggagacaggggcgccttataggtgccattatcggcagtgtagctcttggggttgcaacagctgcacagataacagcggccgcggctctgatacaagccaaccagaacgctgccaacatcctccggcttaaggagagcattgctgcaaccaatgaagctgtgcatgaggtcactgacggattatcacaactagcggtggcggttgggaagatgcagcagtttgttaatgaccagtttaataatacggcgcgagaattggactgcatcaaaattacacaacaggttggtgtcgaactcaatttgtacctaactgagttgactacagtgttcgggccacaaatcacctcccctgctttaactcagctgaccatccaggcactttataatttagctggtggcaatatggattacttgttgactaagttaggtgtagggaacaatcaactcagctcgttaattggtagtggcttgatcaccggcaaccctatactatatgactcacagacccaactcttgggcatacaggtaaatttaccctcagtcgggagcctaaataatatgcgtgccacctacttggagaccttatccgtaagcacaaccaaagggttcgcctcagcccttgtcccgaaagttgtgacgcaagtcggttctgtgatagaagaacttgatacctcatattgtatagaatccgatctggatttatattgtacaagggtagtgacattccccatgtctcctggtatttattcctgtctgagcggcaatacgtcagcttgtatgtattcaaagactgaaggtgcactcactacaccatacatggccctcaaaggctcagttattgccaattgcaagatgattacatgcagatgtgcagatcccccaggtatcatatcgcaaaattacggggaagctgtgtccctaatagataaacactcatgcaatgtcttatccctagacgggataaccctgaggctcagtggggaatttgatgcgacttatcaaaagaacatctcaatactagactctcaagtcatcgtgacaggcaatctcgatatatcaactgagcttgggaatgtcaacaactcgataagcagtgccctggacaaattagcggaaagcaacagcaagctaaacaaagtcaatgtcaacctaaccagcacatctgctctcattacttatattgttctagctgtcatatctcttgttttcggtgtaattagcctggttctagcgtgctacctgatgtataaacaaaaagcacaacaaaagaccttactatggcttgggaacaataccctcgatcagatgagagccactacaagaacatgaatacagatgagagacggatgtatccccagtagcaattcgtgtgtcaattctgatagcctgccaattgggagaattaagaaaaaactactggatataagtgaccaaagagcaatacacgggtagaacggtcggagaagccaccccttagtcaggaaccaggcttcacaacatccgttctaccgcattaccaatagcagacttcagtcatggatcgtgtagttagcagagttgtactagagaacgaagaaagagaagcaaagaacacatggcgcttggttttccggatcgcagtcttatctctaatagtaatgactttagctatctctgtagccgccctggtatacagcatgggggctagcacaccgagcgaccttgcaggcatatcgacggtgatctctaaggcagaggatagggttacatctttactcagttcaaatcaagatgtggtagataggatatataaacaggtggcccttgagtccccgctggcgttgctaaatactgaatctataattatgaatgcaataacttctctctcttatcaaattaatggggctgcaaataatagtgggtgtggggcacctgttcatgacccagattatattgggggggtaggcaaagaactcatagtagatgacacaagtgatgtcacatcattctatccttcagcataccaagaacacctgaattttatcccggcgcctactacaggatcaggctgcactcggataccctcattcgacatgagcgctacccactattgttatactcacaatgtgatattatctggttgcagagatcactcacactcacatcagtatttagcgctaggtgtacttcggacatccgcaacagggagggtattcttttctactctgcgttccatcaatttagatgacacccaaaatcggaagtcttgcagtgtgagtgcgactcctttaggttgtgatatgctgtgctctaaagtcatagagactgaggaggaggattataagtcagttacccccacatcaatggtgcatggaaggttagggtttgacggtcagtaccatgagaaggacttagacgtcacagtcttatttaaggattgggttgcaaattacccgggagtgggaggagggtctcttattgacgaccgtgtatggttcccagtttatggagggctaagacccaattcgcctagcgacactgcacaagaagggagatatgtaatatacaagcgctataataacacatgccccgatgaacaagattaccaagttcggatggctaagtcttcgtataagcctggacggtttggtggaaagcgcgtacagcaagccatcttatctatcaaagtatcaacatctttgggcgaggacccggtgctgactgtaccgccaaatacagttacactcatgggggccgaaggcagagtcctcacagtagggacatctcatttcttgtaccaacgagggtcttcatacttctctcccgccttattataccctatgacagtacacaacaaaacagctactcttcatagtccttatacatttaatgctttcactcggccaggtagtgtcccttgccaggcatcagcaaggtgccctaactcatgtatcactggagtctatactgatccgtatcctgtagtcttccataggaatcacaccttgcgaggggtgttcgggacaatgcttgataatgaacaagcaaggctcaaccccgtatctgcagtatttgattacacatctcgcagtcgcataacccgggtaagttcaagcagcaccaaggcagcatacacgacatcgacatgttttaaagttgtcaagaccaataaagtttattgccttagcattgcagaaatatccaacaccctatttggggaattcaggattgtccctttactggtcgagatcctcaaggatgatagggtttaagaggctaagttcagccagccgggtcaaccacaagagggatgggaagatgacgttgtattacctatcttctgcaatgccaaggatcaagcggaataccaatacgagcccgaatcttatgctatcgagcagccataatcggacaatgctgacatgatcagtttgaatcttgtcaatagtcactctgtttagaaaaaatatgaaaggtggtgagatataagaaaaaacaacttacagaagatagcacgggtaggacatggcgggctccggtcccgaaagggcagagcaccagattatcctaccagagtcacatctttcctccccattagtcaaacacaaattgctctattactggaaattaactgggctaccgcttcctgacgaatgtgattttgaccatctcattatcagcaggcaatggaagaaaatccttgagtcagccactcctgacactgagagaatgataaaactcgggcgggcagtgcaccagactctcaaccacaattctaagataaccggagtgctccatcctaggtgtttagaagaattggctagtattgaggtccctgattcaactaacaagtttcggaagatcgaaaagaagatccagattcacaacacaagatatggagaaatgttcacgaggctgtgcacgcacgttgaaaagaaattgctaggatcatcctggtctagcaatgtcccacgatcagaggaattcaacagcatccgtatggatccagcattctggtttcactcaaaatggtccagagccaagtttgcctggctccatataaaacaagtccaaagacatctgattgtagcagcaagaacaaggtccgcagtcaacaaactagtgacactgactcataagataggccaagtctttgttactcctgagcttgtcattgtgacacatacagatgagaacaagttcacatgcctcacccaggaacttgtattgatgtatgcagatatgatggagggcagggatatggtcaacataatatcttccacggcgacacatctcagaagcctatcagagaaaattgatgatgttctgcggttagtggatgctctagcaaaagatttgggcaaccaggtctatgatgttgtatcactgatggagggattcgcatacggtgccgttcagctgctcgagccgtcaggtacatttgcaggagatttttttgcattcaacctacaggagctcaaagatactctaatcgaactcctcccaaatgatatagcagaatcagtgactcacgcaatcgccaccatcttctccggcttagaacagaatcaagcagctgagatgttgtgtttgctgcgtttgtgggggcacccactgcttgagtcccgtattgcagcaaaagcagtcaggagccagatgtgcgcaccaaagatgctagacttcgatatgatcctccaggtattatctttctttaagggaacaatcatcaatggatacagaaagaagaactcgggtgtgtggccacgtgtcaagatggatacaatatacgggaaggtcattagacagctgcacgctgattcagcagagatctcacatgatgtcatgttgagggagtacaagagtctatctgcacttgagttcgagccatgtatagaatatgaccctgtcaccaacctaagcatgttcttaaaagacaaggcaatcgcacatccaaaagataactggctcgcctcattcaggcgaaaccttctctctgaggaccagaagaaacatgtaaaggaggcgacctcaactaatcgcctcttgatagagttcttggaatcaaatgattttgatccatataaggagatggaatacttgacaacccttgagtacctaagagatgacaatgtagctgtatcatactcactcaaagagaaggaagtgaaagttaatgggcggattttcgctaaattaaccaagaaattaaggaactgtcaggtaatggcagaaggaattctagctgaccaaattgcaccttttttccaggggaatggggtcattcaggatagcatatccttgactaagagtatgttagcaatgagccaactatcttttaacagcaataagaaacgtatcactgactgcaaagaaagggtttcctcaaaccgcaatcatgatccaaaaaacaagaatcgccgaagagttgccacttttatcacgactgacctgcagaagtactgtcttaactggagatatcagacaatcaagctatttgcccacgccatcaaccagctgatgggcctacctcacttctttgagtggattcatcttagactaatggacactacgatgtttgtaggagaccctttcaatcctccgagtgacccgaccgactgtgatctctcaagagtcccaaatgatgacatatatattgtcagtgctagggggggcattgaggggttatgccagaagctatggacaatgatctcgattgctgcaatccaacttgctgcagcaagatcgcattgtagagttgcctgcatggtacaaggtgacaatcaagtaatagctgtaacgagagaggtaaggtcagatgactcctcggatatggtgttgacacagttgcaccaggccagtgataatttcttcaaagagctgattcatgtcaatcatctgattggccataacctaaaagatcgtgaaaccatcaggtcagacacattcttcatatatagcaaacgaatattcaaagatggagcaatactcagtcaggtccttaagaactcatctaaattagtgctgatatcaggtgaccttagtgaaaacactgtaatgtcctgtgccaacattgcatccactatagcacggctgtgtgagaacgggcttcctaaggacttctgttattatttaaactacctaatgagttgcgtacaaacatatttcgattctgagttttccattactcacagctcgcagccagattccaaccagtcctggattgaggacatctctttcgtacattcgtacgtcttaacccctgcccagttggggggactgagtaatcttcaatactcaaggctctacacaaggaacatcggtgacccggggaccactgcctttgcagaggtcaagagattagaagcagtggggttgctgagtcctagcattatgactaacatcttaacaaggccacctggcaatggagattgggccagtctttgcaacgatccatactcctttaattttgagactgtcgcaagcccaaatattgtccttaagaaacatacacagaaagtcttattcgaaacttgttcaaaccccttattatccggagtacatacagaggataatgaggcagaagagaaggcattggctgaattcttactcaatcaagaagtggttcatccacgtgtcgcgcatgctatcatggaagcaagctctgtaggtaggagaaagcaaatccaagggcttgttgacacaacaaacactgtgattaagattgcactgactaggaggcctctcggcatcaagaggttgatgcggataatcaattactcgagcatgcatgcgatgttattcagagatgatgtttttttgtccaacaggtccaaccaccccttagtctcctctaatatgtgttcgctgacgctagcagattatgcacggaacagaagctggtcaccattgacagggggtaggaaaatactgggcgtgtctaatcccgataccatagagcttgtagagggggagattcttagtgtcagcggagggtgcacaaagtgtgacagcggagatgaacagtttacgtggttccatcttccaggcaatatagagctgaccgatgacaccagcaagaatcccccgatgagagtgccatatctcggatcaaagactcaagagaggagggctgcctcgcttgcaaaaatagctcatatgtcacctcatgtgaaggcagcactaagggcatcatccgtgctaatctgggcgtatggggacaacgaagtaaactggactgccgctcttcagattgcaaggtctcggtgcaatataagctcagagtatcttcgactattgtcacccctacctacagctgggaatctccaacatagattggatgatggcataacccaaatgacatttacccctgcatctctctatagggtatcaccttacattcacatatccaatgattctcaaaggctatttaccgaagaaggaatcaaagaggggaatgtggtttaccaacaaattatgctcttaggtttatctctaattgaatcactcttcccaatgacgacaaccaagacatatgatgaaatcacattacacctccacagtaaatttagttgctgtatcagggaagcacctgttgcagttcctttcgagctcctcgggttggcaccagagttaaggacagtaactacaaataagttcatgtatgatcctagccctgtatcggagagagactttgcaagacttgacctagctatcttcaagagttacgagcttaatttggagtcataccccacaatggagctaatgaacattctttcaatatctagtgggaagttgattggtcagtccgtggtttcctatgacgaagatacctctatcaagaatgacgccataatagtgtatgataatacacgaaattggatcagtgaagcccagaattcagatgtggtccgcctattcgagtatgcagcactcgaagtgctcctcgactgtgcttatcaactctactatctgagagtgagaggcctaaacaacatcgtcctatacatgagtgatttatacaagaatatgccaggaattctactctccaatattgcggccacgatatctcaccccatcattcattcaaggttgaatgcagtaggtctagtcaaccatgacgggtcacatcagcttgcagacacagatttcatcgaaatgtctgcgaaactgttagtctcttgcactcgacgcgtggtatcaggtttatacgcagggaataagtacgatctgctgtttccatctgtcttagatgataacttgagtgagaagatgcttcaactgatttcccggttatgttgtctgtacacagtgctctttgctacaacaagagaaatcccaaaaataagaggcctatcggcggaagaaaaatgctcagtactcactgagtacctactgtcagatgctgtgaaaccattgcttaggtccgaacaggtgagctctatcatgtctcccaacataattacattcccagccaacctatattacatgtctaggaagagccttaacttaatcagagaacgagaggacagagataccatcttggcattattgttccctcaggaaccgctacttgagtttcgtccattacaagatattggtgcgcgagtgaaagatccatttactcgacaacctgcagcgttcatacaggagttagatttgagtgctccagcaaggtacgatgcatttacacttgatgagtttcaccttgagcacacactgccgaacccagaggaagattacttagtacgatacttgttcagaggaatagggactgcttcatcttcttggtataaggcatctcatcttctttctgtacctgaggtcagatgtgcaaggcatggaaactccctatacttggcggaaggaagtggagccattatgagtcttcttgaactgcatataccgcacgagaccatctactataatacgcttttctcaaatgagatgaaccccccacagcgacatttcggaccgactccgacacagtttctaaattcggtcgtttataggaatctacaggcagaagtgccatgtaaggatggatttgtccaggagtttcgccccttatggagagagaatgcagaagaaagtgacctgacctcagataaagcagtagggtatatcacatctgcggtgccatacagaactgtatcattactacattgcgacattgaaattcctccaggatccaatcaaagcttactagatcaactggctaccaacttatccctgattgccatgcactctgtaagagagggcggtgtcgtgatcatcaaagtattgtatgcaatgggatactacttccatctgctcgtgaatttattcactccttgttccacaaaagggtatattctctctaatggctatgcctgtagaggggatatggagtgttacctgatattcgtcatgggctatgtaggcggacctacattcgtgcacgaagtggtgagaatggcaaaaactctaatacagcggcacggtacgcttctgtcaaaatcagacgaaattacgttgactaggttatttacctcacagcagcatcgtgtaatggacattttatccagtcctttaccgagactgatgaaattcttgagagagaacattgatgctgcattgattgaagccggggggcagcctgtccgtccattctgtgcagagagtttagtgagcacactaacagatatgactcagatgactcagatcatcgccagccacattgatacagtcattcgatctgtgatctacatggaagctgagggtgaccttgctgacacagtgttcttatttactccttacaatctttctacagacggtaaaaagagaacatcacttaaacagtgcacaagacagatcctagaggtcacaatactaggtcttagagtcaaagatctcaataaagtgggtgatgtaataggcttagtactcagaggtacagtttctctggaggatctcattccactgaggacatatttgaagcgtagtacctgcccaaaatacttgaaggcagtcctgggtattactaaactcaaagaaatgttcacagacacctctttattatacctgactcgtgctcaacaaaaattctatatgaaaaccataggcaatgcggccaagggatattacagcaataatgattcctaaaggcgatcacatatgaataggttctcttcctagccaactgtgtcctcataaacctgatcataccgtattagaaaaaagttgaattccgattctttggaactcgtattcggattctaataattatctttaaacggaagtatgcgtagttgttcttgactatagtcctgtcattcaccaaatctttgtttggt

SEQ ID NO: 2 - DNA, 15780 bp, Newcastle Disease Virus
accaaacaaagatttggtgaatgacgagactacactcaagaataattgtgcgcaacctttttttaagacatttatttgagttcgaattcgagtcctaaggagtcagggttcaacttttttctaacataatatgattaaatcaacaagaatacaattggccaaaaaaggagcctattaatatgtgattttcgttaagagtcacagttactgtaatatcctttgactgcattgcctatagttttcatgtagaatttttgttgagcacgagtcaagtacaatacagaagtgtctgtaaacatttctttgagtttggtaatacctaggacagccttcaaatatttagggcaggtactatgcttcaagtatgtccttagtgggataaggtcctccatggagatcatgcctttaagcactaggctgattatatcgcctattttattgagattttcgactctaagacctagtattgtaacctctaggatctgtctcgtgcactgtataagtgatgtcctctttttcccgtcagtagagagattgtaaggggtaaatagaaatactgtgtcagcgagatcaccctcagcttccatatatatcacagaccggataactgtgtcaatgtgactagcgataatctgggttatctgagttatgttcgctagcgtgctcaccagactctccgcacagaatggacggacgggctgtcccccggcttcaatcagcgcagtgtcaatattcttcctcaagtactttattaatcttggtaaaggactggataggatgtctgtcacacgctgccgctgtgaggtgaataacctggtcagtgtgatctcatctgatttagacaagagcgtaccgtgccgctgcaccagagtttttgccatcctcaccacctcatgtacaaatgtaggcccgcccaggtaacccatgacaaataccaggtaacactccatatctcctcgacatgcataaccattagagagaatatatccttttgtggaacacggagcaaacaagttcatgagtagatgaaagtagtatcccattgcatacaacactttgatgattactaccccgccctcccttacagaatgcatggcaatcagagataaattgatagctagttgatctagtaagctttgattggaccctggaggaatttcaatgtcacaatgcagcaatgatacagatctgtagggcactgcagatgtaatataccccactgctttatctgaggtcaggtcactttcctctgtattttctctccataatggacggaactcttggacaaatccatctttgcatgttacctccgcctgtagattcctataaacaaccgaattcaaaaactgagttggggtcggcccgaaatgtcgttgcggggggttcatctcatttgaaaagagcgtattgtaatagatagtttcatgtggtacatgcagttcgagaagactcatgatggctccgctcccttcagctaagtataaggagttcccgtgtcttgcacatcttacctcgggtacagaaaggagatgagatgccttataccaagaggaagatgcagtccctatccctctgaacaagtatcgtactaagtagtcttcctccggatttggagatgtgagttcaggatgaatctgactaagtgtgaatgcgtcataccttgctggagcactcaaatctaactcttgcaaaaatgccgcaggttgtcgggtgaatggatctttcactcgagcaccaatatcttgcacagaagggaactctaataatggctcttgggggaacaacaacgccaggatagtatccctgtcctccctttccctgatcaaattgaggctcttccgagacatgtagtacagattagctgggaatgtaattatgttaggagacatgatagagctcacttgatcggggctaagtaatggtttcacagcatccgacagtaaatactcagtgagtattgaacatttctcttctgcagttaagcctcttattttcgggatttctcttgttgtagcaaagagtaccgtgtacagacagcataaccgggatatcagctgaagcatcttctcattcaggttatcatctaagacagatgggaacagcagatcatatttatttcctgaatataagccggagatcacacgtcgggtgcaagatactaatagttttgcagacatttcgataaaatccgtatctgcaagttggtgtgatccgtcatggttgaccaggcccactgcatgtaaccttgaatgaatgacgggatgagatattgtagctgcaatgttggaaagtagaattcctggcatattcttgtataaatcacccatatataagacaatattgtctaggcctcttactctcaggtaatagagttggtaagaacagtcgaggagcacttcaagtgctgcatattcaaataggcggaccacatctgaattctgagcttcactgatccaatttcgggtattgtcatacactattatggcgtcattctttatggaggtatcttcatcataagaaaccacagactggccaatcaacttcccgctggatattgaaagaatgttcattagctctatcgtgggatatgactccagattaagctcataactcttgaagatagctaagtcaagtctcgcaaagtctccctccgatacagggctaggatcatacataaacttatttgaggtcactgtcctcagttccggtaccaccccaagtagctcgaaaggaaccgcaacaggtgcttctctgatacagcaactaaatttactatgtaggtgcagtgtgatctcatcatatgtcctggttgttgtcattggaaagatcgattcgattagagataaacccaagagcatgactctgttggtaaaccacattcccctctttgactccttcttcagtgaacagcctttgagaatcattggatatgtgaatgtaaggtgacacctgtagagagatgcaggggtgaatgtcatctgagttataccatcatctagtctatgttgaagattcccagccgtgggtaaaggggacagtaaccgaagatactctaagtttacattacaccgagattttgcaatcgtaagagcagcagtccaatttacttcattatccccataagcccagatcaacacggatgatgcccttagggcagcctttacatgtggcgacatatgagctatttttgcaagtgaggcagctctcctctcctgtgtctttgacccgagatatggtaccctcatcggaggattcttgctggtgtcatcggtcaattctatattgcttggaagatggaaccaagtaaattgttcatctccgctgtcacatcttgtacaccctccgcttacactaagaatctcaccctctacgagttctatcgtatcaggattagatacacccagtattttcctgcctcccgtcaaaggtgaccagcttctattccgtgcatagtctgccagtgtcagagaacacatattagaagagactaaggggtggttggatctactggaggaaaaaacatcgtctctaaacagcattgcatgcatgctagaataattgactatccgcatcagcctcttgatgcctaatggcctcctagtaagcgcaatcttaattacggtgtttgttgtgtcaacaagcccttgaatttgctttctcctacctacagagcttgcctccatgatggcatgcgcaacgcggggatgaatcacctcttgattaagcaagaattcagccaatgccttctcttctgcctcattatcctctgtgtgcactccagacaataagggatttgaacaagtttcaaataggactctttgcgtatgtttcttaagaacaatatttgggcttgcaacagtctcaaaattgaaagagtatgggtcgttgcacagactggcccaatctccattcccaggcggcctagttaagatattagtcataatgttaggactcagtaatcccactgcttctagtcgcttgatctctgcaaaagcagtagtccccgggtcaccgatatttctagtgtagagccttgagtattgaaggttactcagtccccctaattgggcaggagtcagaacatatgagtgcacaaaagagatgtcctcaatccacgactgattaagatcggggtgcgaattgttggtgatggagaactcagagtcaaagtatgtctgcacacaactcattatatagtttaaatagtaacagaagtctttgggaagcccgttctcgcatagccgtgctacagtagaggcaatgttggcacaggacattacggtgttttcactgagatcacctgacactagcactaatttagatgaatttttgaggacttgactgaggattgctccatctttgaagattcgtttgctgtatatgaagaatgtgtctgacctgatggtttcacgatccttcaaattatggccaatcaaatgattgacatgaattaattccttgaagaaattatcactggcttgatgcaactgtgtcaacaccatctccggagagtcgtctgatcttacctctctcgttactgctattacttgattatcaccctgtaccatacaggcaacacgacaatgcgatctagctgcagcaagttggattgcagcaattgagatcattgtccatagcttctggcataatccttcgataccccctctggcactgacaatatatatgtcatcattagggactcttgagaggtcacagtcagtagggtcacttggaggattgaaagggtctcctacgaacatcgtagtgtccatcagtcttaggtgaatccattcgaagaagtgaggtaggcccatcaactgattgatggcatgagcgaacaatttgattgtctgatatctccaattaagacagtacttttgcaggtcagttgttatgaaggttgcaactctccgacggttcttgcttttcggatcatgattgcggtttgaagatactctttctttacagtcagtgatacgtttcttattgctattaaaagacagttgactcatcgctagcatactcttggtcaaggatatgctatcctgaatgactccatttccctgaaagaaaggtgcaatctgatcggctaggatcccttccgccatcacctgacagttccttaacttctttgtcagcttagcgaagatccgtccattaactttcacttccttctccttgagcgagtatgatactgccacattgtcatctctaaggtactcaagggtcgtcagatattccatctctttatatggatcaaaatcatttgactctaaaaactctatcaagaggcgattagtcgaagttgcttcttttacatgtttcttctggtcttcggagagaaggttccgcctaaacgaggcaagccaattatcgttggggtgtgcgattgccttgtcttttaggaacatgctcaggttggtgacagggtcatattctatacatggctcaaattcaagtgcagataaactcttatactctctcaacatgatatcgtgtgaaatctctgctgaatctgcatgtagttgcccaatgaccttcccatatattgtatccactttgactcgcggccacacacctgcattcttctttctgtacccgttgatgattgttcccttgaagaaagacagtacctgaaggatcatatcaaagtctaccattttcggtgcgcacatttggctcctgactgcctttgctgcattacgggactcaagcagtgggtgaccccacagacgcaacagacacaacatctcagctgcttgattctgttctaaaccagagaatacagtagcgattgcatgagtcacggattctgctatatcattggggaggaggccaattagaatgtccttaagctcctgcaggttgaatgcgaagaaatctcctgcaaatgtacctgacggctcgagtagctggacagctccgtatgcaaatccctccattagtgatacaacatcgtagacttgattacccaagtcttttgccagagcgtctattaaccgcaaaatgtcatcaattttctctgataagcttctgagatgcaccgccgtggttgatattatgttgaccatatctctgccctccatcatatctgcatacatcaatacaagttcctgggtaagacatgtgaacttgttctcattcgtatgcgtcacaacgacaagttcaggagtgacaaagacttggcctaccttatgggttagcatcaccaatttgttggccgcagaccttgtcttagctgccaccatcagatgcctctggatctgttttatatggagccatgcaaacttggctgtggaccattttgagtgaaaccagaatgccggatccgtacgaatgctgctgaactcctctgaccgggggacattgttagaccaagatgaccccagcagtttcttctctatatgcgtacacagccttgtgaacagttctccatatctcgtgttgtgaatttggatcttcttctcaatcttccgaaatttgttggttgaatctgggacctcaatattagccagttgttctaaacacctggggtggagcactccggttattctggaattgtggttaagagtttggtgtactgcccttccgagttttatcattctctcagtatcaggagaggccgattcaagtatttttttccattgtcggctgagaatgaggtggtcgaagtcacattcatcaggaagcggtagcccagttaatttccagtaatagagtagtttgtgcttgaccaatggtgaagacaggtgtggctctggtaggataatctgatgctctgccctttcaggaccggagctcgccatgtcctacccgtattatttaccatgagctgttttgccttgtatctcattgccacttacattttttcttaatcaagagattaatgacaaggcttaatctgatcgcatgagcactggctgattgtggtcaactggcaacatggaattcgagcacgcgccggcattcggtttgattcttgatgtcgcagaagataggtgatacaatgccatctttccaactcctttataattgactcaactagccagacctggcttctctaaccccgtcatctttgaggatctcaactagtaacgggacgattctgaattctccgaagagagtattagatatttcagcaatgctgagacaataggtcttattagtcttgaccactttaaaacaagttgatgttgtgtatgctgctttggtactgcttgaactcactcgagtaatgcgactgcgggatgtgctatcgaatactgcagacgcagggttaagtcttgcttgtacaccatcaagcattgtcccgaatacccctcgcaaggtgtggtttctatagaagattaggggatatggatctgtatagactccagtaacacacgagttggggcatcttgctgaagcctggcaagggatactacctggccgagtgaaggcattgaatgtataaggactatgaagagtggctgttttgttgctgactgtcataggatataataacgcgggagagaagtatgatgaccctcgttgatacaagaaatgagatgtccctactgtgagaattctgccttcggcccccatgagtgtgactgtgttgggcggtacagtcagtaccgggtcttcgcctaaggatgttgacaccttgatagataagatagcctgctgtatgcgtttcccaccaaaccgtccaggcttatacgaagacttggccattcgaatctggtagtcttgctcatctgggcatgtgtcattgtatcgcttgtatatcacatatttcccttcctgtacagtgtcactgggtgaattgggttttaaccctccgtagactgagaaccatacgcggctgtcaataaaagatccaccccctactcctgggtagttggccacccagtccccgaataatgttgtgacatctaggtccttttcgtggtactggccgtcgaaccctaacctcccatgtaccatccgcgtagggacagctgagttataatcttcttcctctgtctccgtgactttcgagcacagcatatcacaacccaggggagttgcactcacactgcaagacttccgattttgggtgtcgtccaggttgatggaacgcagagtagaaaagaataccctccctgtcgcagatgtccggagcacaccaagtgctaaatactgatatgaatgtgagtgatctctacatccagacaatattacattatgggtgtagcagtaatgggtagcactcatgtcaaatgagggtattcgagtgcaacctgatcctgtagtaggcgccgggataaaattcagatgttcttgaaatgcagagggatagaatgatgtgacatcactagcatcatctacaatgagttctttgcctatcccccctatataatctgggtcatggataggtgccccccacccactgttgtttgcagctccattaatctgataagagagagatgttattgcgttcataattgtggtctcagtatttaacaatgccaacggagactcaagggccacttgcttatatatcctatctactacatcttgattggaaccaagtgtagatgtaatcttttcttctgccctggaaatcctagtcggtatgcctacaagatcgctaggtgtgctagcccccatgctatataaaagggaggctacagatatagccaaggtcactactgttaagaataagattgcaatccggaatatcaagcgccatgtattttttgcctctctttcatcattctctaacgcaacttggctaacggcgcggtccatgattgaggactgttgtcggtgaagcggtagaacggaggttgtgaagcctggctcgcaattgaggggcggcctctcttaccgttctacccgtatatcgtcctttggtcatctacaaccggtagttttttcttaactctctgaactgacagactaccagaactttcacacaaattactattagggaaaccttcgttcctcatctgtgttcacatttttgtagtggctctcatctgatctagagtattattcccaagccataataaggtcttttgttgcgccttttgcttgtacattaggtagcatgctagaatcaggctaagtataccaaaaacaagagatatgatagtcaaaacgatataggtaatgagagcagatgtgctagtcagtttgacattgactttgtctagttttctgttgctttcctctaacttattcaaagcattactgatcgagttgttgacattcccaagctcagttgagatatcaagattgcctgttattattacttgagaatcttgtattgagatattcttctgataagttacatcgaattccccactgagccttaaagttatcccgcctaaggataaaacattgcatgattgtttatctattagagacacggcttctccatagttttgcgatatgatacccggggggtttacacatctacatgttgtcatcttgcagttggcgatgactgaaccttttgtagtcatgtatggtgtagtaagtgcgccttcggtctttgagtacatacaggccgacgtattgccgctcaagcaggaataaataccaggggacatagggaacgttactattcttgtacaatataaatctaagtcagtttctatacagtatgaggtgtcaagttcttctatcacagaaccgacctgtgtcaccactttggggacaagtgccgaggcaaatcccctggttgtgcttacggataaggtttccaagtaggtggcacgcatattatttaggttcccgactgaaggtagagttacctgtatacccaagagttgagtctgtgagtcgtatagaatagggttaccggtgattaagccgctaccgattaatgagctgagttgattgttccctacacctaacttagtcaataagtaatccatatttccaccagctagattgtaaagtgcctgaatagtcagcttgtttaaagcaggtgaagtgatttgtggtccgaatactgtagtcaattcggttaggtacaggttgagctctacaccaacttgctgtgcaattttgatgcagtctaattcctgagctgttttattaaatcggtcattaacaaactgctgcatcttcccaactgccactgctagttgcgataatccgtcagtgacctcatgcacagcctcattggttgcggcaatgctctctttaagtcggaggatgttggcagcattttgtttggcttgtatcagagctgcggccgctgttatttgtgcggcagttgcaaccccaagagccacaccgccaataatggcgcctataaggcgcccctgtctcccccctccagatgtagtcacagactcttgtatcctacggatagagtcaccaaggggggtgagcaaagtggtcaatgtcctgttgtatgcatccaagggggctttcgcacatgcctccttatccttgggcagattcgggaggagcttaactatgattgatcctgtctgggatgaggtgtatatgttgacggctttgtctcctgtaaccacaattcctgcagctgcaagaggcctgccatcaatggagtttgccggacagatgcaactcagtaccagcgcaacccggatagtcagcatcataggtgctgggttcttggtagaaggtctggagcccatcttgcacctggagggcgccaaccgggatccagaatcttctacccgtgttttttctaacttgatagacaggtaaactaactgtaaataatcaagacagattagttttttgtgtcatgatgatctgggtgagtgggcggagcgcaatctcagagacgcagcttatttcttaaaaggattgtatttggcaagggtgtgccccttctccagcttagtagaggtaacctcgtggtcggcggtcactgcgacagcgcgttgggtacctgcttggataatgattttaacgctccgcaggcacgcggtttgactccagagtatcttggccacctgaggagaagcatttgctatgggatagcaggctgtcccactgctagagaagaaaggtgccaaaagcttagtccgtgcacctcttgcttttaccaacacggaaggcccgagcacatcactgagcccgacagatagatcaaggctccttattttcttttccagcttgtcaaatgtcactttcttccccttcctatctacggtggtcataagtccaatatgcaagaagaggttagcatagtatccgctgtcagacttagacaaagatttaaccaaaggactcctcgggtctacctccacattaatagtgacattgagcgcaagattgtacagactcgagccagaaaccttcaatactgcagctgggatcttgtagacatccttcttcggtaccacagtcaaggagacaaagttcaccttgtattctagggttccactcccgggaatcttctctggtgctttcacgtgcttgactgcattcactgatgagtatttgtttgccacaaccctacagctttgcagcacttggggtgcctgcactactgagaaaaccattctctcagtattagttgcactcttcttgcatgtgactatcatagtgagacaggcccttgccagctcaataaggtatccggtatttgggacgcttcctaggcagagcatcgcagcggaaagtaactcgcgcttgggtttatcatcgatcatgccgacagtggcttcttcattcccaacttgaaagatgaatccataggtggtgatgaatactgagtcctccttactatcagtccacaagtcaaggcgctggatcctatattgcggggcgatttgcttcttcccatctcctgtgccttgtaggacgatcggaaatgctaacaggttgctagaagaatgggcagaatcaaagtacagcccaattgtcctagatgagtccatcttggcacaattggggcactccaattctacccgtattttttcttaatcttaaatgtagctagattaattacggtttcgaaatggatccagtttaaacccattagcaattgcatctcctctgagactggctccttttccttttgaaactcggaggatccaccagcttctgtaagatgctgaagagttcattcgcggctttgcgctggattctcaagtcgttcatcgggagcttggccaggtccatgatatctttcaccttcttcacgccatcaggaaggttgtttttcagagtatagagctcctcagatatgtgtttgatgtgcggctttgacttgtcagtgttttcaagcatttccaagtacatcgaaacaatctggctcagtatgatccttttctcatttctctctgtccagttcttcagtttctctacaataataggtccaccgtcagctacatctgaatgacttgagttaaagtcagctttcagtttgtctatatcatcttgaagttgaacaagatttagactacttgcagtatgtccataataaatcatgatgacagacagaacaaacaagttgtaagtctggcaagccatggtggcagaaggatcagcgcttcgatcttctacccgtattttttcttaatccgcggttgcatcgatcgcgtcaatttcgaccattcagtggggctgaggaagcgagagaggattgccgcttggagagttggaccttgggtctgcgggggggaactcggtgcagattccgtgtgatgccgagtggacagggacccgctacgtgtggcagtagtaattagccatttagagcaaggcgcttgattttcctgatttcctcgatcgacccggctgcatctaacttgcttaggagcttggctgaagaactcgggtgcattgggcgtgacatgatcaatgcacggacagtgtccttttccactcctatatcaggcccgcatgcagtggcgggtttaatcaattcagatggatgtggcactggttgcgaaagtttattaagtgccatttcgcctccttgtgtcacatagggagaggggtctccagggcctgaaactaaaaccgggtgagatcgggcaactgcccgtagatcactcagagatgaaatgttggcacaaccgggatccagaatcttcatcattcccaagttggcttccatgactgcaacagatgttttcagctgttggatttcggaccgcatcatagggatggaggatgtctgtttcaagacaagatctagctgatagtcaaccttacttactctctgtgatatcgcctccatcatagacatcatcgcttgcacaaagtctacaggtggctggacatgatccgcagatacaagggtattgtcttggctctgccttgatcggagagcatgaggggttgcaccagctgatagttgtgactcctcccattgtccatgatatgctgtgttcacgtctgtgccctggtttccaggggcggccttgacttggttctgcggtctttcctgactgtttccgcgactgggttgactcccctgctgttgagtcggacgttggtgattcccctcttgggggctcgaccatgggccctttttagcattggacgatttattgctgagcttgtcaagcatcaacagcagagagttgcttgctccggtcctgaactgtgtgtcaacggcttcgtctgtggcctgggtggggggctggtcggcggatgtggccggcgggctgtcatgcggggtcgtttgctcgggtgtggatggttgtttgtcagatctgtcctgtcgatcggggttgtcttgactggccggtggctggatgctcccatgcttctcccatgctgcgctcagcaccttggtcttgccttgtgggattgcactccttccaacagtctctgctggtttaccctgggctgtaattatgttgtcaatgacagttccacttgtctcaaatagctcgtcgatctctgcatctgtaaaggtggccatgtttgtcctggtctatcaggtagaggagagagcagagactcgggagacttgccctgatctctgaatatccctcttctacccgtacttttttctaattccctcggctctgttttgattgttagtgagccgcactgtgcctgtggtatctagggcgtgcggagttgtacagcagggggagggaggaaagagggggatgtttgtttgagggtgtggtcatatagagccgcaaatcgaggggtcgactacgggtgagggcattgggatgtttttgtggaagcaggctgggttttgtccatcaatacccccagtcggtgtcgttatcttgggatggcccaggagttgggggaggccccgattggggagtgccctgtgcagagtttggcgcctccctcatgctatttgctaccgctctcatcagatccaggaattgggtctccccatccccggcttctggttgcccttgcgatatattcgatccgccttgtagagcttgggaccccccctcgctaagcccagtgaggactcggatttgttgagtaggcatgtatatgctgctggtatcgtcggagacccgttgggcagcagctgccaggcccttcattgctgctggggttagctttagctcggcagccatatcctcgttaatgctacttccctgagcctgagcgtactctactccaagtctccagaatgatgtgctcataaagtccctggcaaattggtatttcccagtacctttatctaggactgatgccatacccatggcaaaggagtaaagttgtgcatactcggcaggcgcaaagctcatctggtcactatcaccaagtaatgtcatgtacggcgcattatctcctttcatccgatacaaacgcatgagctgcttcatcttctggatgtcgcctgagaggctactaagtgcaagggctgatgtcttggtgttgattccgtacttgagtgtcaagaagaatgcagtaagcccggtattcctgatgtatgagtctacgtcccctaccaggttataataagtagaggtaccacctgccgtgttgcggcctctcttgagctcgctaaccaaaaagatgcggactgcaagagactgtctgatcgtgagttggattgtgctcctgcatacggggtagaggatgtatttcttttggaccctgccttgctgcatatacttattgattcgccttgtttccgactcatctgcagtctcatacgcagtcatggcttttgctactgtgacccatacttgagcctggatagagaggatcctctccagggtatcggtgatgtcttctggtgcatcatcttctgccccggctgtgacgaacggggttccgttgctgcatgcccgagggagagatcctgctatcatcgcaaatctctgtgctctctcttcagacactccactcctattgttgaactggggcgtgccgttggcaaagccatcaatctcaagcacggccaatgtggcttcattctgtttccctgcaatggcaacatggttcctcattacctgtgagtgggagcataaaagagatatgagagcaccttgcctgagtggtttgttggcatcttcgctaacagcaatccggaggcagaataccacaaagctccatctatcttctgggtcatcactgttaagagtgaataccgggacgtctacttttaaggtactccctttttctccccctccatgagctccattggggcgagtctgagccgcgaggagctgttcgtactcatcaaatacggaagacatgttggcagaaggctttctcgagtttgtgcttcgggctcgcactcgagattcacaccttctacccgtgcgacttcaattgctccttcgccttttatcgtaactcacggattctctgtttggt

SEQ ID NO: 3 - DNA, 20 bp, Artificial Sequence
  Oligonucleotide
tttcttaatcttaaatgtag

SEQ ID NO: 4 - DNA, 18 bp, Artificial Sequence
  Oligonucleotide
cggattatcgcaactagc

SEQ ID NO: 5 - DNA, 20 bp, Artificial Sequence
  Oligonucleotide
tcaccatcacaacctacaac

SEQ ID NO: 6 - DNA, 20 bp, Artificial Sequence
  Oligonucleotide
cagggcactttgattctacc